(19) United States
(12) Patent Application Publication
(10) Pub. No.: US 2019/0175895 A1
(43) Pub. Date: Jun. 13, 2019

(54) VIBRATION SYSTEM LOCALIZED PROXIMATE A TARGET ARTERY

(71) Applicant: Parallel Biotechnologies, LLC, Miami Beach, FL (US)

(72) Inventors: Arkady URYASH, Miami Beach, FL (US); Andrew Kenneth HOFFMAN, Burnaby (CA)

(73) Assignee: Parallel Biotechnologies, LLC, Miami Beach, FL (US)

(21) Appl. No.: 15/945,689

(22) Filed: Apr. 4, 2018

Related U.S. Application Data
(63) Continuation-in-part of application No. 13/992,092, filed on Jun. 6, 2013, now abandoned.
Application No. 13/992,092 is a continuation of application No. 13/986,252, filed on Apr. 17, 2013, now abandoned.
Application No. 13/986,252 is a continuation-in-part of application No. 12/798,437, filed on Apr. 5, 2010, now Pat. No. 8,870,796.
Application No. 12/798,437 is a continuation-in-part of application No. 12/291,128, filed on Nov. 5, 2008, now abandoned.
Application No. 12/291,128 is a continuation-in-part of application No. 12/218,054, filed on Jul. 11, 2008, now Pat. No. 8,734,368.
Application No. 12/218,054 is a continuation-in-part of application No. 11/036,386, filed on Jan. 18, 2005, now abandoned.
Application No. 11/036,386 is a continuation-in-part of application No. 10/902,122, filed on Jul. 30, 2004, now Pat. No. 7,517,328.

(30) Foreign Application Priority Data
Sep. 4, 2003 (CA) 2439667 A1

(51) Int. Cl. A61M 37/00; A61B 8/08; A61N 7/00; A61H 23/00; A61B 17/22; A61H 19/00; A61H 23/02

(52) U.S. Cl. CPC A61M 37/0092; A61B 8/4455; A61N 7/00; A61H 23/00; A61B 8/483; A61B 17/22004; A61H 19/32; A61B 8/0816; A61H 23/0236; A61H 19/50; A61H 19/34; A61H 23/0263; A61H 2201/1619; A61H 2230/505; A61H 2201/165; A61B 2017/22001; A61B 8/08

(57) ABSTRACT

The present invention relates to a wearable (or fastenable, or adherable) vibration massage system directly localized, focused and engaged upon or overlying a selected target artery of a user. The vibration massage waves are advantageously derived from religious texts, whereby a user can simultaneously listen to audio of the in real time, thereby providing a synchronized, harmonized tactile and auditory experience. The provided system is essentially MUSical vibration applied upon a target ARTtery—hence is hereinafter described as the “MUSART” therapy system. Various modes of application of the tactile vibratory stimulus, as well as modes for invasive delivery of acoustic stimulation directly in contact with a target artery, are presented.

### PRIORITY CLAIM AND INCORPORATION BY REFERENCE

The present application is a continuation in part of co-pending U.S. patent application Ser. No. 13/999,092 filed on Jan. 13, 2014 which is a continuation in part of U.S. patent application Ser. No. 13/986,252 filed Apr. 17, 2013 which claims priority to U.S. patent application Ser. No. 12/798,437 filed Apr. 5, 2010, now U.S. Pat. No. 8,870,796, which claims priority to U.S. patent application Ser. No. 12/291,128 filed Nov. 5, 2008 which claims priority U.S. patent application Ser. No. 12/218,054 filed on Jul. 11, 2008, now U.S. Pat. No. 8,734,368, which claims priority to U.S. patent application Ser. No. 11/036,386 filed on Jan. 18, 2005 which claims priority to U.S. patent Ser. No. 10/902,122 filed Jul. 30, 2004, now U.S. Pat. No. 7,517,328, which claims priority to Canadian Patent Application No. 2439667 A1 filed Sep. 4, 2003. The contents of these applications are incorporated herein by reference in their entirety. All references cited herein are incorporated by reference in their entirety.

### FIELD OF THE INVENTION

This invention relates to non-invasive systems for imparting vibration, and more particularly mentally or cognitively stimulating, and preferably religious text musically derived tactile vibration massage applied locally proximate a target artery or arterial vasculature of an individual with synchronized listening. The invention provides improved athletic and mental (including sexual) performance, cardio and neurologic protection, pain management, treatment for ischemic conditions, arthritis, and expedited anti-inflammatory, healing and regenerative effects.

### BACKGROUND OF THE INVENTION

Musical Vibration Therapy (MVT) is a recently recognized technology that uses music (for listening), coordinated with tactile vibration massage (with oscillation rhythms and/or waveform frequencies which match and/or are harmoniously coordinated to some degree with the musical rhythm and/or waveform frequencies), that are applied directly to the body. The technology has historically employed speakers or transducers placed within mats, mattresses, chairs, recliners, tables, or soft furniture, generally taken together with an audio speaker for correlated, in synch listening (provided by head phones for example) to provide a combined, synchronized tactile and auditory experience. Established benefits include pain management, relaxation and anxiety relief, improved blood flow, musculoskeletal physical therapy, treatment for arthritic conditions, and general health improvement.

Various vibration/sound properties can be manipulated to attain specific physiologic and psychological experiences.

A patient's enjoyment of music (or alternatively any other cognitively meaningful, or mentally stimulating audio waveform), in addition to correlated (or derived) tactile vibration massage, plays a key role in motivating use of MVT. The majority of patients are interested in using MVT because it is a treatment modality that is pleasant, entertaining, distracting and comforting, unlike many invasive and potentially unpleasant medical procedures.

From a biomedical standpoint infrasonic to sonic, or “audible” frequency mechanical vibration massage is a known potent blood flow stimulator, largely because the transmitted vibrations induce sheer stresses within the vasculature which lead to an endogenous liberation of beneficial mediators such as endothelial Nitric Oxide (NO) which is a potent vasodilator, and Tissue Plasminongen Activator (TPA) which prevents abnormal clotting. Vibration coupled with enhanced NO release in turn leads to a synergistic relaxation of the smooth muscles within the lining of arteries and arterioles (through promoted actin—myosin sarcomere decoupling), which decreases arterial spasm (common in many ischemic conditions) and generally diminishes vascular tone which promotes blood circulation. Low frequency, tactile audio stimulation also importantly provides enhanced endothelial Nitric Oxide Synthase (eNOS) bioavailability through elevated expression of eNOS mRNA which can warrant sustained enhanced blood flow to select organs as a lasting effect. Moreover, vibration is known to induce other beneficial molecules, thereby promoting development of new collateral circulation to ischemic areas, or even healthy areas which may benefit from additional blood flow.

Vibration massage in the sub-audible to audible frequency ranges has therefore enormous potential applications in treating ischemic pathology (acute or chronic), as well as bolstering the vascular system in normal subjects for increased mental and physical (including sexual) performance, whereby MVT with correlated listening plays an important role in promoting use of the therapy by making it fun, pleasurable and entertaining.

Olav Skille, in U.S. Pat. No. 5,101,810 disclose a MVT system, however the provided therapeutic equipment for applying tactile therapy (speakers) are in all cases embedded with a bench, bed or chair, which cannot importantly focus the musical vibration with high efficiency, towards or directly into a patient's vascular system (which is preferred to maximize vascular sheer stresses which lead to endogenous liberation of beneficial mediators to optimize blood flow stimulation effects). Moreover, it would be most desirable to provide an MVT system which could be made portable and be worn as a garment (or alternatively underneath or alongside one's clothing, or disposed as jewelry) or adhered onto the skin surface to expedite use of the therapy during normal everyday activities. Also, the '810 patent's disclosed modes of downloading selected musical tracks (for both listening and correlated vibration massage) are outdated, and require a more accessible modern framework including network, social app ecosystem and wearable device integration.

Vulfson in US patent application US 20090180646 disclose a wearable musical system comprising a “tactile subwoofer for accurate reproduction of sound waves” which may be applied as a “part of a garment of clothing, or a clothing accessory, or a personal accessory or jewelry” with correlated listening. However the '646 publication is not prescribed for therapy (i.e. the device is only designed to promote and accentuate listening appreciation of a user) and hence has no provisions to enable targeted focusing of the therapy with correct positioning and engagement force located towards and upon an arterial system of a user, which is required to provide and ensure therapeutic and performance enhancing blood flow stimulation effects.

U.S. Pat. No. 6,638,295 B1 to Frederikus Schroer teaches a mechanical pressure application device that applies constant pressure to compress an extracranial blood vessel. The pressure is merely constant mechanical pressure and is not variable not in response to any musical or vocal input.

Published U.S. patent application US 2007/0038164 A1 to Shariar S. Afshar teaches an apparatus and method for improving a user's interaction with multimedia content using a vibration system arranged on the pectoralis major muscle group. The primary function seems to be improve the sensation of low frequency sounds for entertainment purposes. No therapeutic uses are taught.

Published U.S. patent application US 2007/0067054 A1 to M. Sherif Danish teaches an apparatus and method for assisted learning such as of a language or religious text. The apparatus does not contain any means of transferring vibration to a user other than sound waves to the ears.

U.S. Pat. No. 4,630,519 to Mutsuo Hirano et al. teaches a vibrator device which produces rhythmic pulses corresponding to a beat pattern of a music rhythm. There is no teaching of the use of religious text in this application. Nor is there any teaching of health benefits from using the device.

The prior art has therefore failed to provide a wearable network integrated MVT system which can with a high confidence and efficiency target and focus therapy directly into the arterial vasculature of a user to provide optimized systemic and localized therapeutic blood flow stimulation effects. Furthermore, the prior art has failed to describe means for locating and maintaining (as well as providing appropriate engagement force upon) a tactile vibration site on a body surface generally overlying a target artery to enable such therapy, nor biofeedback or instrumental means for verifying or ensuring a target blood vessel is being effectively oscillated. Moreover, the prior art has not considered variations of imparting MVT therapy invasively, directly within the human body. Further the prior art has not taught that the use of religious text has a synergistic effect when used in the present device. As such, there remains a need for an effective MVT therapy.

### SUMMARY OF THE INVENTION

The present invention relates to a new and improved wearable or fasten-able system for delivery of MVT therapy whereby the musically derived tactile massaging component is directly localized, focused and engaged (with appropriate engagement force) upon a selected target artery of a user. The provided MVT system is essentially MUSical vibration applied upon a target ARTtery—hence is hereinafter described as the “MUSART” therapy system.

The preferred embodiment comprises a small portable and wearable (i.e. fasten able and/or adhere able) therapeutic mechanical oscillation transducer (hereinafter “MUSART therapeutic transducer”) configured to enable targeted, localized placement upon a selected “target” artery or arterial system of a user, which once placed provides a cognitively meaningful, emotionally and/or intellectually (or otherwise “mentally”) stimulating oscillation waveform, such as derived from a musical audio track or optionally other cognitively stimulating acoustic sound sources (hereinafter MUSART sound source), directly, and with a high degree of efficiency towards and into the target artery.

The MUSART therapeutic transducer obtains a “mentally stimulating” input waveform with distinct frequency, wave-shape and amplitude characteristics, preferably derived from music, to emit a corresponding (i.e. with matching, in—synch rhythm, wave-shape and frequency) mentally stimulating tactile oscillation to the body surface. The mentally stimulating oscillation (or percussive or vibratory massage waves by other name) thereby oscillate, and preferably to some degree compress and decompress the target artery, to provide an optimized pleasurable and entertaining tactile response, as well as a sheer producing, beneficial vascular response to a user.

The MUSART system is also fundamentally equipped with means for a user to “listen” and be entertained by the correlating or coordinated musical (or otherwise mentally/cognitively stimulating) waveform which serves as the template for the derived vibratory tactile massage. To this end the MUSART system also comprises a set of head phone speakers—hereinafter MUSART headphones—(although if discretion is desired, ear buds, or an external speaker may alternatively be used) operable with the MUSART therapeutic transducer for added temporally in-synch listening of the contents of the mentally stimulating MUSART sound source waveform in combination with the tactile sensation. To be clear, “in-synch” listening means that the emitted “audible” waveform emitted by the MUSART headphones (for listening) is temporally matched in frequency, tempo and wave-shape with the massaging oscillation waveform (for tactile response) emitted from the MUSART therapeutic transducer.

A selected target artery to receive MUSART therapy is preferably palpably superficial relative to a skin surface of a user (e.g. radial artery, brachial artery, femoral artery, abdominal aorta, popliteal artery, pedal artery, tibial artery, or carotid artery) but in some applications may also be relatively “deep” (e.g. coronary artery, cerebral artery—including the temporal arteries), however in all cases the selected target artery must be anatomically locatable to a reasonable degree of accuracy beneath a selected external body surface by means of non-invasive inspection, to enable targeted, localized placement of the MUSART therapeutic transducer over or generally proximate the target artery.

Methods for locating a body surface position overlying a target artery for positioning of the MUSART therapeutic transducer comprise at least one of; arterial palpation (e.g. by use of a finger of a user), arterial heat, Doppler flow, arterial derived motion (detectable by at least one of a heat sensor or anemometer, Doppler flow sensor, or force sensor respectively), or simply by observation of an external body surface anatomic landmark which is known to correlate with a target arterial position. Palpation of a selected target artery (when available, such as with relatively superficial target vessel) is generally the preferred method, as it is easiest and cheapest.

Methods for maintaining a body surface position overlying a target artery in the MUSART method comprise simple visual inspection that the MUSART therapeutic transducer has not migrated from its original position. This is easily accomplished in most cases as an engagement face of the MUSART therapeutic transducer (or alternatively an “applicator face” which acoustically couples the MUSART therapeutic transducer to the selected body surface, described later) has a strategic surface dimension orient able with respect to the short axis of a selected target artery which is at least 1.5 times a typical diameter of the selected target artery. Thereby, maintained acoustic coupling of the MUSART therapeutic transducer upon or proximate a target artery is ensured regardless of slight movements or migration of the MUSART therapeutic transducer during use. Alternatively, an arterial “sensor” (such as arterial heat—preferably an anemometer, Doppler flow, or accelerometer) may be non-invasively disposed alongside the MUSART therapeutic transducer (i.e. whereby both the MUSART therapeutic transducer and the “sensor” are orient able along the long axis of the target artery) to provide a more exacting means for monitoring that the MUSART therapeutic transducer is remaining upon the target artery during the course of therapy.

The MUSART therapeutic transducer preferably comprises an audio speaker emitting mechanical oscillations within the infrasonic to sonic range (i.e. 1 Hz to 20 kHz), being housed within a resilient sound case having a circular oscillation emission face of about 1.5 cm in diameter (which is greater than or equal to 1.5× the diameter of most palpable peripheral arteries while being advantageously small enough—i.e. less than about ¼^{th} a length of the applied body surface—to enable focussed directed arterial compressions rather than, and somewhat less effectively, a nonspecific shaking of the target artery with the surrounding tissue). A variety of acoustic transmission engagement face “applicators” (which are in acoustic contact with the emission face of the speaker and enable transmission of vibrations generated by the speaker to a body surface of a user), are also detachably and interchangeably provided in a range of shapes and textures with varying elasticity and malleability to enable contoured and comfortable seating against a variety of selected body surfaces. Alternatively, the oscillation emission face of the speaker may also be used directly on a patient's body surface without an applicator.

Intellectually and emotionally (or mentally) stimulating sound waveform signals are stored within a plurality of selectable MUSART sound source audio tracks, with each sound track having a particular value for treating a differing pathology or effecting a variety of therapy goals. Each MUSART sound source audio track contains the required audible waveform for processing to enable tactile massage by the MUSART therapeutic transducer coordinated with in-synch listening by MUSART headphones. Audio track information is stored (and is modifiable by a physician or qualified staff) on the Internet or social media network, for easy downloading and streaming to/via either tablet or smart-phone using cellular, WiFi or NFC technology, providing easy access.

MUSART tactile massage therapy is further particularly effective when administered at a displacement waveform frequency lying in or about the resonance frequency range of an underlying target tissue, organ or vasculature (which usually correlates to the 1 Hz to 300 Hz, or “bass” frequency pitch range)—which thereby maximizes the internal oscillatory responses to the non-invasively provided vibration. Lower frequency bass waveforms are also generally preferred as such lower toned frequencies can be emitted at relatively higher displacement amplitudes (or stroke lengths) relatively safely and comfortably to the human body, to provide a more forceful oscillation felt and transmitted to the body surface and thereby to the target artery. Higher frequency pitched tones (e.g. greater than about 300 Hz) if delivered at comparably high displacement amplitudes may for example cause unwanted tissue heating and likely pain to a user. The bass frequency vibration waves will likely also, as music comprises a broad range of varying bass frequency, occasionally directly engage an advantageous tissue or arterial resonance frequency for added momentary synergistic internalized vibratory (and sheer producing) effects.

Therefore it is preferable in MUSART therapy to accentuate the musically derived bass tones (i.e. <=300 Hz), versus the higher pitch tones of an inputted musical piece emanating from the MUSART therapeutic transducer, to promote tactile vibration massage emissions with waveforms which tend to match tissue or internal organ (or vascular) resonance, which commonly reside in the 1 Hz-300 Hz, and usually at least 8 Hz, and most commonly in the 20 Hz-120 Hz range. Hence it is a preferred feature in MUSART therapy that the bass frequency aspect of a utilized “mentally stimulating” audio source waveform for tactile massage applications, by use of a processor (hereinafter “MUSART processor”), may be utilized alone (whereby higher pitched tones are filtered out by a low pass filter), or most preferably in most applications be relatively amplified or increased in amplitude with respect to the higher pitched tones as emanating from the MUSART therapeutic transducer.

The MUSART therapeutic transducer by use the MUSART processor is also advantageously enabled to emit a complex vibratory tactile massage waveform, whereby the inputted musical or other mentally stimulating oscillation waveform from the MUSART sound source may be advantageously blended with (or “amplitude modulated” by) a selected distinct “base frequency waveform”, preferably comprising a sine wave with a selected base frequency, although other wave-shapes such as square—or percussive wave, triangular or saw-tooth, or other programmable wave shapes may be used. Essentially, the user would, in this preferred embodiment, experience a smooth periodic increase and decrease of tactile massaging intensity (comparable to tactile “volume changes”-which might be felt to the skin surface like a fluttering), at a frequency correlating to the selected base frequency. The base frequency may, for tactile enjoyment reasons, be most preferably blended in synch with and matching (or alternatively comprising a multiple of) the cadence or rhythm (or tempo) of the musically derived inputted waveform, while also, for therapeutic reasons approximating an established resonance frequency of a target tissue or vasculature.

Furthermore to ensure emission of a fairly continuous stream of tactile vibration massage waves with emission waveform frequencies at or near tissue resonance even during moments of correlating “silence” or low volume during a musical piece, the oscillations derived from the base frequency waveform (as discussed above) may also be delivered continuously or near continuously during times of such musical tone “silence” or low amplitude. From a user's perspective, this generally preferred embodiment of MUSART therapy would feel like a gentle regular massaging effect during moments of musical silence (e.g. via an emitted sine wave, which has amplitude peaks which are preferably in-synch with—and match or alternatively comprise a multiple of—the rhythm frequency—or equivalently cadence, beat frequency or tempo—of the musical piece). So in this preferred embodiment of the invention, a user would feel a regular tactile massaging sensation with displacement amplitude peaks which are temporally synchronized to the musical beat during times of musical silence (like a synchronized drum roll sensation), and then would feel the melodic musically derived waves of varying frequency and wave-shape (also preferably being amplitude modulated by the base frequency waveform) during the moments when the music manifests.

From a biomedical perspective, upon entering the body the frequency components of a “base waveform” blended with a musically derived oscillation waveform become demodulated, so the tissue targets receiving vibration massage will experience and benefit from the frequency components of both waveforms (i.e. derived from music and distinct base waveforms) simultaneously. Sine waves as base waveforms (particularly for amplitude modulation of the musically derived waveforms) are generally preferred because they comprise pure tones with precisely matched increase and decrease of amplitude and having a smoothly rising and falling curvature exactly replicated with each cycle. These pure tones are particularly useful because they do not produce overtones that could adversely affect the coordinated musical vibration experience.

To enable hands free engagement of the MUSART therapeutic transducer fasten-able (and preferably wearable) upon a selected body surface overlying a selected target artery, a series of MUSART inflatable sleeves (or bands, or rings by other name), available in varying sizes (to accommodate varying sized individuals) are provided, which encircle a limb, extremity, appendage or trunk of a user (e.g. arm, forearm, wrist, leg, foot, hand, hip, abdomen or penis). Each sleeve disposes and secures a MUSART therapeutic transducer to enable for targeted localized application upon at least one of the brachial, radial, femoral, aortic, popliteal, tibial, pedal or penile arteries. MUSART sleeves are preferably cosmetically ornamented to resemble jewelry, and/or may provide another useful function commonly required by a user, such as an arm band of a wrist watch for radial artery applications.

MUSART sleeves are preferably automatically inflatable upon touch of a button to at least 40 mm Hg and up to about 100 mm Hg (or nominally 80 mm Hg) of pressure such as to match a typical physiologic (i.e. life sustaining) arterial pressure, and preferably a diastolic arterial pressure of a user. Pressurized engagement force of the MUSART therapeutic transducer against a target artery, at or near diastolic pressure greatly accentuates the vibratory transmission and vascular oscillatory response (particularly enabling partial alternating compression to decompression of the target artery) when an applied MUSART therapeutic transducer begins to oscillate. MUSART sleeves are preferably equipped with an automated pressure regulator, to automatically induce and maintain diastolic pressure (once activated) as sleeve pressures may be prone to change due to positioning changes on the body part applied (e.g. along the wrist or forearm), or due to morphologic or size changes to the body part treated during therapy (e.g. penile stimulation—discussed later).

To enable carotid arterial oscillations (which provide therapy to the carotid arteries, but also by transmission to the cerebral arteries and brain tissue), the MUSART therapeutic transducer is preferably in most cases simply placed over a carotid artery (within the “carotid triangle of the neck of a user) by use of a double sided adhesive tape, which is fairly inconspicuous and also works very effectively, particularly if the user sits back or reclines (so the weight of the MUSART transducer can provide a degree of engagement force). Alternatively, a regular tape may be used to overly the MUSART transducer which assists engagement force of the unit against the carotid artery. In a more elaborate variation a specially adapted collar (resembling a neck brace) may be provided place-able about the neck of a user which enables disposition of a MUSART therapeutic transducer upon the anterior-lateral aspect of a patient's neck (such as to overly a carotid artery) without adversely effecting the breathing of the user.

MUSART therapy to the carotid artery is particularly useful for the indications of emergency treatment of acute ischemic stroke (whereby the vibration waves and pressure fluctuations are transmitted from the carotid artery to the cerebral arterial vasculature to help clear thrombus), physical and occupational therapy application to improve cerebral flow in a recovering stroke victim (whereby the vibration waves stimulate vasodilation and induction of cerebral arterial angiogenesis), and a vascular healing application of the carotid artery following vascular trauma, such as in a stent procedure (e.g. to limit inflammation to reduce the risks of re-stenosis and late in-stent thrombosis).

To enable therapeutic MUSART therapy to the coronary arteries, which is particularly useful for the indications of; treatment of refractory angina (by promoted coronary angiogenesis or arteriogenesis), emergency treatment of heart attack (by assisted clot disruption), pre-ischemic conditioning prior to coronary stenting or bypass surgery (by enhanced NO bioavailability), and improved coronary artery healing post stent procedure (by reduced inflammation in prevention of re-stenosis and late in-stent thrombosis), two methods may be considered.

First, as the coronary artery is a relatively deep, non-palpable vessel, an operator may place a variant, higher powered MUSART therapeutic transducer (in this case preferably comprising a linear stepper motor which enables high fidelity, controllable tactile MUSART vibratory waveform emissions at comparatively high stroke lengths—e.g. 0.1 mm-4 mm)—over at least one brachial artery of a user, whereby the oscillation induced compressions and decompressions of the brachial artery and vibrations along the arterial wall provide a battery of hemodynamic fluctuations (including pressure, flow and volume waves) retrograde through the aorta to provide therapeutic sheer producing effects to the coronary vasculature.

A specially adapted, preferably inflatable MUSART sleeve which enables engagement forces of the variant high powered MUSART therapeutic transducer upon the brachial artery in the 40 mm Hg to 100 mm Hg (nominally 80 mm Hg) range, is provided to enable hands free engagement of the transducer against the applied body surface of the arm. A biofeedback sensor which in this case preferably comprises a finger plethysmograph (to sense propagating hemodynamic fluctuations in blood volume arising from the brachial artery), or alternatively at least one of an anemometer (e.g. DermaFlow differential thermal analysis technology), Doppler flow probe or force sensor (i.e. accelerometer) disposed upon a carotid artery of the user, is used co jointly with tactile brachial arterial MUSART therapy to ensure the brachial artery is being therapeutically oscillated, and serially compressed and decompressed.

Second, a similarly powered variant MUSART therapeutic transducer (again preferably comprising a linear stepper motor enabling generation of high fidelity tactile musically derived MUSART vibration massaging waves) may be placed directly to the chest wall (or alternatively upper back) overlying the heart of a user, for the same above described purposes, whereby in this case the tactile oscillation waves are transmitted transthoracically from the rib-spaces of the chest wall (or alternatively upper back) directly to the coronary vasculature. Oscillation displacement amplitudes of at least 1 mm, and preferably 2 mm to 6 mm are required, with significant engagement force applied upon the variant MUSART therapeutic transducer upon the chest wall (e.g. at least 10 Newtons, preferably 20-50 Newtons) to enable sufficient vibratory penetration to effectively reach the coronary circulation. Again, finger plethysmography, or alternatively an anemometer, Doppler flow or force sensor disposed upon a carotid artery (all of which can show propagating, vibration induced hemodynamic fluctuations measurable from the carotid artery) can provide extra assurance that the provide tactile vibratory waves are effectively providing vibration to the myocardium and coronary arteries thereupon.

Means for hands free engagement of MUSART tactile therapy upon the rib-spaces of the chest wall include a wearable vest—or more preferably a tighten able elastic belt assembly which attaches with the variant MUSART therapeutic transducer and once worn by the user provides the required engagement force of the device upon the chest wall, (while still allowing the freedom of the user to breath normally) during reception of MUSART tactile therapy. An optional means for applying MUSART tactile therapy to a chest wall surface is by the hands of the user (i.e. by self administration) or by an operator, as engagement force can be thereby controlled—and preferably titrated to a maximal level of patient tolerance (which will vary markedly based on the constitution and sensitivity level of the patient).

Engagement applicators for chest wall MUSART application targeted to the coronaries preferably comprise a plurality of contact nodes spaced relative to one another to enable seating upon the anatomic left and right sternal margins of the 3^{rd} and 4^{th} intercostal spaces (or to the left and right of the spine of a user)—which have been shown by the Applicant to anatomically match and overly the location of the left and right coronary arteries. Alternatively, and most preferably, the engagement applicator may comprise a pair of gel pads place-able to the left and right of the sternum including the ribs and intercostals spaces, which once compressed with sufficient engagement force enable automatic countering of the chest wall surface and efficient transthoracic transmission of oscillatory energy. Direct vibration overlying the sternum (i.e. oscillation of the sternum) may also be employed (as bone is a particularly good acoustic transmitter), although this technique may be painful to some users.

To enable therapeutic MUSART therapy targeted to the arteries of the male external genitalia, a specially designed elastic ring and optionally harness (both of which house a MUSART therapeutic transducer) is adapted for placement around in the first case (ring) the penile shaft and in the second case (harness) the scrotum for targeted application (depending on the placement and orientation of the ring/harness) to at least one of the dorsal artery, deep central artery, cavernous artery, bulbo cavernous artery, internal pudic artery, inguinal or cremasteric arteries. Daily exposure of MUSART therapy to the penile and cremasteric arteries, particularly when base frequency musical amplitude modulation with a continuously applied base frequency tone is added, enhances penile and perineal blood flow potential (which provides over time a larger, maximum size of the penile head and shaft), and provides enhanced NO bioavailability stored within the penile tissue to promote lasting and sustained erections during intercourse.

The MUSART therapeutic transducer (via ring and/or harness assembly), may optionally also be worn by a user during intercourse, and when the therapeutic transducer is oriented on the superior aspect of the erect penis or scrotum provides excellent stimulation to the male dorsal, perineal and cremasteric artery (thereby promoting blood flow and sustained erection to the penis), while (in the ring embodiment) simultaneously providing the potential for erogenous tactile stimulation to the female clitoris and G spot. It is thereby conceived as a preferred feature of this embodiment of the invention that both a male and female may co jointly listen to a selected musical piece which is harmonized and in-synch with and utilized as the sound source for the tactile massage emissions for the penile vasculature.

The substantially circular nature of the penile MUSART applicator (transducer plus ring) enables vibration emission predominantly from the transducer, but also by transmission from the ring itself (which doubles as an engagement means from the transducer, and an engagement face applicator for the transducer), hence simultaneous exposure of vibration to a number of the penile arteries occurs simultaneously regardless of exact position of the ring, which may be moved or shifted periodically from its resting location, particularly during intercourse. In a deluxe variation, the penile ring is also made inflatable, and can be anatomically maintained at or near physiological pressure (i.e. in the 40 mm Hg to 100 mm Hg range, nominally 80 mm Hg), via a microprocessor and pressure gauge regulator operable with the ring shaped applicator. Maintenance of engagement force of the penile MUSART therapeutic transducer against the shaft of the penis in approximation of a user's diastolic blood pressure advantageously enhances penile blood retention while safely allowing systolic flow (for increased penile size during sexual performance) and also maximizes therapeutic vibratory response of the penile arteries emanating from the MUSART therapeutic transducer.

To enable therapeutic MUSART therapy to the female external genitalia a specially designed clip applicator which disposes the MUSART therapeutic transducer is provided, adapted for placement against the clitoris glans (or more preferably, for added comfort to the hood or prepuce of the clitoris) for targeted massage application to the clitoral arteries (i.e. the deep and dorsal clitoral arteries) as well as, by transmission, the pudendal and inguinal arteries. Daily exposure of MUSART therapy to at least one of the clitoral arteries, particularly when base frequency amplitude modulation and a continuously applied tone is added, enhances clitoral blood flow potential and NO bioavailability to promote enhanced sexual arousal, performance, and enjoyment (including multiple orgasms) during intercourse.

Due to the non-invasive application of the clitoral clip, the applicator may also be advantageously worn during intercourse or sexual foreplay, and may be worn discretely throughout the day or at parties per the discretion of its user. The clip is preferably manufactured in one size with a nominal clasping tension against the body surface which approximates a physiological pressure (i.e. again in the range of 40 mm Hg-100 mm Hg), with the body surface pressure varying depending on the size of the clitoral glans or hood of the user.

To ensure and monitor appropriate and maintained positioning of a MUSART therapeutic transducer upon a selected superficial, “palpable” target artery, a biofeedback arterial heat sensor (preferably an anemometer) is optionally provided being disposed alongside the therapeutic transducer (both therapeutic transducer and heat sensor being disposed along the long axis of the target artery), to enable confirmation that the therapeutic transducer is over the underlying artery. Alternatively, a Doppler flow or force sensor (e.g. an accelerometer) may be used, or coupled with the anemometer, to confirm arterial pulsatile flow or volume pulsations which also confirm an artery's location. In this variation, a visual or audio signal is advantageously displayed to alert a user that the MUSART therapeutic transducer is well placed, or has moved and hence is no longer transmitting vibration directly over and upon the target artery. Alternatively, the biofeedback sensor may be disposed within, or about the therapeutic transducer according to the invention, which while more expensive a configuration may provide means for more exacting therapeutic arterial placement. In yet another variation, for applications where a stronger, higher displacement amplitude massage transducer is utilized, at least one of; an anemometer, Doppler flow sensor, accelerometer or a plethysomograph disposed to an artery or body part remote from the target artery (to sense transmitted arterial hemodynamic fluctuations arising from the target artery) may also be employed to assess for appropriate target arterial acoustic stimulation.

However it should be understood that the addition of such biofeedback arterial locator systems comprise a deluxe feature of the MUSART system which are not absolutely required to enable the MUSART method, as target arterial location by palpation and/or through simple inspection of anatomic landmarks usually provides reasonably accurate placement of the MUSART therapeutic transducer over a target artery, whereby maintained engagement may be informally confirmed by the user, by periodic visual inspection.

Finally, it is also conceived in the present invention that a MUSART therapeutic transducer may be surgically inserted invasively into the human body, placed to a location within the soft tissue generally proximate, and in acoustic contact with a selected target artery. In this invasive variation, various arterial parameter sensors may be included disposed proximate and operable with the MUSART therapeutic transducer (such as described for the non-invasive applications) to ensure proper and safe function of the device in acoustically stimulating the selected artery. The invasive MUSART therapeutic transducer is preferably made non-invasively rechargeable with parallel electromagnetic coil technology (a commercially available technology, e.g. “Millar wireless charging system”), and is adapted to receive programming instructions by way of telemetry (similar to pacemaker technology).

A first, primary object of the invention is to provide a method and apparatus enabling the disposition of a wearable tactile therapeutic oscillation transducer, enabling generation of vibration derived from a cognitive, intellectually and/or emotionally (or mentally) stimulating sound waveform such as preferably music, towards and upon or overlying (or generally proximate) a target artery of a user, to provide a means for promoting blood flow both locally and systemically, for both chronic and acute conditions, as well as to improve mental and physical (including sexual) performance in healthy subjects.

A second primary object of the invention is to provide means for coupling use of the therapeutic oscillation transducer with an audio speaker—either by head phones, ear phones, ear buds or otherwise—to enable a harmonized, temporally in-synch correlation of mentally stimulating listening pleasure to a user in accordance with the corresponding emission of tactile oscillation pulses emanating from the therapeutic transducer.

A third object of the invention is to equip the therapeutic oscillation transducer with processing means enabling a relative accentuation of amplitude for the lower bass frequencies (versus higher pitched frequencies) of an emitted musically derived (or more generally mentally stimulating) tactile vibration waveform.

A fourth object of the invention is to equip the therapeutic transducer with processing means enabling blending of a base frequency oscillation waveform (preferably sinusoidal with a frequency at or near target tissue resonance, and having a frequency, or amplitude peaks, matching the cadence or tempo of the correlating musical piece) which preferably serves as an amplitude modulator to the emitted musical tactile vibration waveform (to effect periodic intensity changes), and is also most preferably utilized as a distinct continuously applied massaging tone (particularly during times of musical silence) to ensure vibration emissions are occurring continuously throughout therapy.

A fifth object of the invention is to provide means for hands free engagement and fastening of the therapeutic transducer upon a selected target artery, preferably via at least one of: a series of inflatable sleeves (equivalently bands, rings or belts) available in varying sizes—for the arteries of the limbs, extremities, trunk, hip region and male genitalia of the body-; an adhesive tape or collar (for the neck region overlying the carotid artery), a′ rib-space chest wall (or upper back) interface with elastic tighten-able belt for coronary arterial applications (along with a special applicator which enables contouring of a chest wall surface and multi-ribspace engagement to the anatomic left and right of the sternum), and a clip or an adhesive (for the arteries of the female genitalia) of the body.

A sixth object of the invention is to provide at least one of a biofeedback sensing or instrumental means to confirm and ensure correct positioning of a tactile therapeutic transducer upon a target artery, whereby the biofeedback sensing means comprises at least one of; a heat sensor—preferably an anemometer, a Doppler flow sensor or an accelerometer disposed adjacent to the therapeutic transducer (whereby both the therapeutic transducer and biofeedback sensing means has an orientation disposable along the long axis of a selected artery to provide direct information on target arterial location relative to the position of the therapeutic transducer), or at least one of; a Doppler flow sensor, an accelerometer or a plethysmograph disposed to an artery or body part remote from the target artery (to sense transmitted arterial hemodynamic fluctuations arising from the target artery). The instrumental means preferably comprises a therapeutic transducer engagement face (or applicator surface) with a dimension sized at least 1.5 times the diameter of a target artery, and preferably less than or equal to about ¼ a dimension of the applied body surface, to ensure focussed localized transducer contact is maintained in acoustic connection with the target artery regardless of subtle movements of the transducer/applicator relative to the applied body surface.

A seventh object of the invention is to provide updated means for downloading or streaming selected musical or gaming tracks, applicable for selected types of therapy session, directly from a social media network via Internet, tablet, smarphone or gaming platform.

An eighth object of the invention is to provide an invasive MUSART therapy solution, whereby a MUSART therapeutic transducer is surgically implanted proximate and in acoustic contact with a selected target artery.

A ninth object of the invention is to provide a set of instructions enabling an optimized method of use of the MUSART therapy system, comprising a paraphrase of the methods described in the forthcoming detailed description. A tenth object of the invention is to provide a list of indications where MUSART therapy may be useful for either the cure or prevention of disease processes, or means of enhancing human performance, as well as information to which target artery should be utilized to achieve such results.

### BRIEF DESCRIPTION OF THE DRAWINGS

The apparatus and method of the present invention will now be described with reference to the accompanying drawing figures, in which:

FIG. 1 is a block diagram flow chart showing the basic constructs of the MUSART system, comprising a mentally stimulating audible waveform source which inputs mentally stimulating waveform information to a processor, which thereafter inputs processed mentally stimulating waveform information to a therapeutic transducer (for tactile administration to a target artery) and unprocessed mentally stimulating waveform information to an audio speaker (for correlating listening to the ear of a user), according to the invention.

FIG. 2 is a perspective view of a patient enjoying MUSART therapy with a therapeutic transducer fastened by an inflate-able band of a wrist-watch directly upon the radial artery, with a gaming track selected for treatment of generalized arthritic pain, according to the invention.

FIG. 3 is a perspective view of a patient enjoying MUSART therapy with a pair of therapeutic transducers fastened by an inflate-able pressure sock upon his pedal and tibial artery, with a gaming track selected for enhanced healing of a broken leg, according to the invention.

FIG. 4 is a perspective view of a patient recovering from a disabling stroke and a carotid stent procedure enjoying MUSART therapy with a therapeutic transducer with triangular shaped applicator applied by adhesive tape upon his recently treated carotid artery, with a relaxing musical track selected for enhanced mental acuity, improved stroke recovery and enhanced carotid arterial vessel healing, according to the invention.

FIG. 5 is a perspective view of a patient prior to undergoing Coronary Artery Bypass Surgery enjoying MUSART therapy with a variant higher powered therapeutic transducer applied by a pressure sleeve upon his left brachial artery (with a finger plethysmograph used to assess for propagating brachial arterial compressions and decompressions), with a musical track selected for cardiac pre-ischemic conditioning according to the invention.

FIG. 6 is a perspective view of a patient suffering from refractory angina having just undergone a coronary stent procedure receiving chest wall MUSART therapy with a therapeutic transducer applied via a tightened belt elastic band assembly with a pair of vibration gel pads disposed at the left and right of the sternum at the level of the third and fourth intercostal space (to overly the basal aspect of the left and right coronary arteries), with a musical track selected for inducing coronary angiogenesis and enhanced coronary arterial vessel healing according to the invention.

FIG. 7 is a perspective view of a male suffering from erectile dysfunction employing MUSART therapy applied to the dorsal artery of the penis, with a musical track selected for inducing enhanced blood flow and increased NO bioavailability to the penis according to the invention.

FIG. 8 is a perspective view of a female enjoying MUSART therapy applied to a clitoral artery (with the therapeutic transducer in this case placed by a clip overlying the clitoral hood), with a musical track selected for inducing enhanced blood flow and increased NO bioavailability to the clitoris according to the invention.

FIG. 9 shows a pair of graphs (with displacement amplitude representing the vertical axis, and time representing the horizontal axis) of; (top) an emitted musically derived oscillatory waveform for tactile massage whereby the relatively lower frequency bass tones have been amplitude enriched relative to the higher frequency tones, versus (bottom) its correlating, in synch audible oscillatory waveform, or (“musical piece”) for correlated listening which shows relatively lower bass frequency amplitude relative to the higher frequency tones, according to a preferred feature of the invention.

FIG. 10 is a graph view of an emitted musically derived oscillatory tactile massage waveform (with displacement amplitude representing the vertical axis, and time representing the horizontal axis) whereby a sine wave amplitude modulation has been added to an otherwise mono-amplitude bass frequency musical tone, according to a preferred feature of the invention.

FIG. 11 is a graph view of an emitted musically derived oscillatory tactile massage waveform (with displacement amplitude representing the vertical axis, and time representing the horizontal axis) whereby a base frequency sine wave oscillation waveform has been added during a time of correlated musical tone silence, according to a preferred feature of the invention

FIG. 12 shows a pair of graphs (with displacement amplitude representing the vertical axis, and time representing the horizontal axis) of; (top) an emitted musically derived oscillatory waveform for tactile massage with base frequencies relatively amplified and an added base sinusoidal waveform emitted during a time of correlated musical silence; versus (bottom) the correlating, in-synch audible oscillatory waveform, or (“musical piece”) for correlated listening which shows a temporally matching frequency and wave-shape. Note how the distance between peaks of the tactile sinusoidal waveform (top) are synchronized as a multiple (in this case double) with respect to the tempo of the musical piece for added harmonious tactile and listening pleasure (bottom). The timing of musical beats are shown as dots on the figure.

FIG. 13 shows an active MUSART therapeutic transducer placed invasively proximate a target artery with an electromagnetic charging coil according to a variation of the invention. Endothelial oscillations with production of intraluminal force vectors (yielding therapeutic endothelial stimulation) is shown.

FIG. 14 is a drawing showing a cell culture apparatus for delivering sound into the culture.

FIG. 15 is a bar graph and corresponding electrophoretic gel showing increased protein upregulation in vitro in the presence of religious texts versus pop music.

FIG. 16 is a drawing showing the structural elements of a cell affected by tactile vibration.

FIG. 17 is a drawing show the pathways affected by the present invention.

FIG. 18 is a photograph showing a rat being treated with the present invention.

FIG. 19 is a bar graph and corresponding electrophoretic gel showing increased protein upregulation in vivo in the presence of religious texts versus pop music.

FIG. 20 is a drawing showing placement of oscillators to represent a laying of hands in the Christian faith.

FIG. 21 is a drawing showing placement of oscillators to represent where bells might be worn in the Hindu faith.

FIG. 22 is a drawing showing placement of oscillators to represent where bells might be worn in the Muslim faith.

FIGS. 23A and 23B are drawings showing placement of oscillators to represent where bells might be worn in the Jewish faith.

### DETAILED DESCRIPTION

The present MUSART invention comprises a tactile vibration system with a provided oscillatory massage waveform derived from an “intellectually and/or emotionally” (or “mentally” stimulating, or “cognitively meaningful”) sound track, preferably from music or optionally video games or other audio related entertainment technology.

A MUSART therapeutic oscillation transducer advantageously emits a tactile, mentally stimulating vibratory waveform in a localized and targeted manner directly upon a body surface overlying a “target” artery of a user (to focus transmission of the oscillations directly into a user's vasculature), while the user simultaneously enjoys a temporally correlated, in-synch harmonized listening of the mentally stimulating, or cognitively meaningful (and entertaining) sound track, of which the mentally stimulating tactile vibrations are derived.

The preferred embodiment of application of the MUSART system to varying body parts for varying purposes is herein below described, along with variations, in reference to a detailed account of the following figures.

In Reference to FIG. 1, a block diagram flow chart showing the basic constructs of a MUSART therapy system is shown. A “Mentally Stimulating Audible Waveform Source” (top box) correlates to a MUSART sound source, preferably comprising a musical sound track, and preferably stored and accessible from the Internet and social media network. Audible waveform information from the MUSART sound source is thereafter downloaded or streamed from the Internet (typically by smart phone, tablet, or game platform technology) to a MUSART “processor” (second box), whereby the audible waveform information is either processed or left substantially un-processed. In the former case, the processed audible waveform information is inputted to a MUSART “therapeutic transducer” (third box, left) and in the latter case, the un-processed audible waveform information is inputted to a MUSART “audio speaker” (third box, right). Finally, the MUSART therapeutic transducer emits tactile oscillations which acoustically stimulate a selected target artery, while the MUSART audio speaker co-incidentally emits audible oscillations which reach the ear of a user, for correlated listening. Importantly, tactile oscillations are temporally synchronized (in frequency—or “pitch”, and wave-shape) to the audible oscillations, to provide a harmonized tactile response with correlated listening experienced by the user.

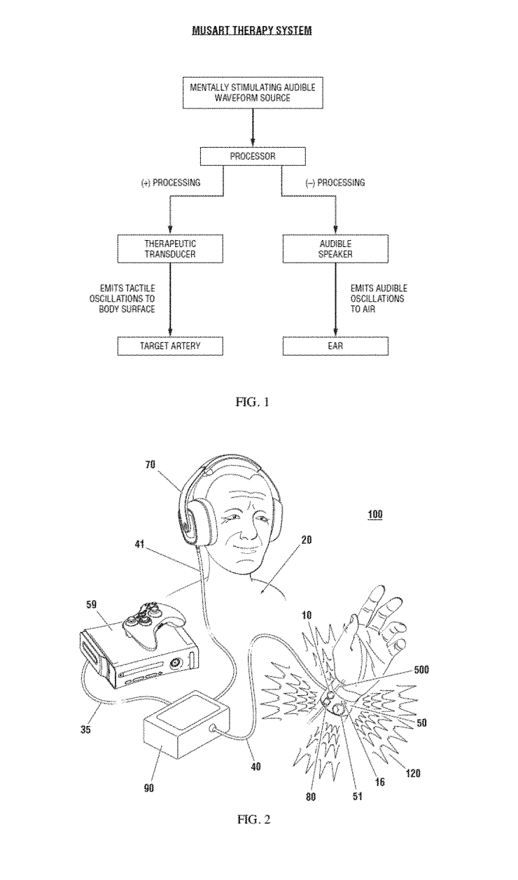

In Reference to FIG. 2, a perspective view of a patient 20 enjoying use of a MUSART therapy system 100 in a preferred embodiment for treatment of arthritis is shown.

MUSART therapeutic transducer 10 comprising a small audio speaker housed within a resilient sound case providing musically and gaming derived oscillations in the infrasonic to audible frequency range (i.e. 1 Hz-20 K Hz), is secured by an inflate-able pressure sleeve 50 (inflate-able elements, not shown, located on the contra lateral wrist surface with respect to the radial artery). Pressure sleeve 50 doubles as an arm band of wrist watch 51, which automatically inflates to a nominal diastolic pressure of 80 mm Hg to provide targeted placement and engagement force of therapeutic transducer 10 upon radial artery 500 (shown as under the skin by dashed lines) of patient 20. A musical track selected for treatment of generalized arthritic pain is down loaded to a MUSART processor 90 from a MUSART sound source (an internet application—not shown) via use of gaming platform 59 and inter-connecting processing cord 35, whereby this information is processed, and fed from processor 90 to the therapeutic transducer via an application cord 40, to emit musically derived vibratory waves (shown as a series of oscillatory wave-fronts 120 emanating from inflate-able sleeve 50, which have originated from the left radial artery target application site).

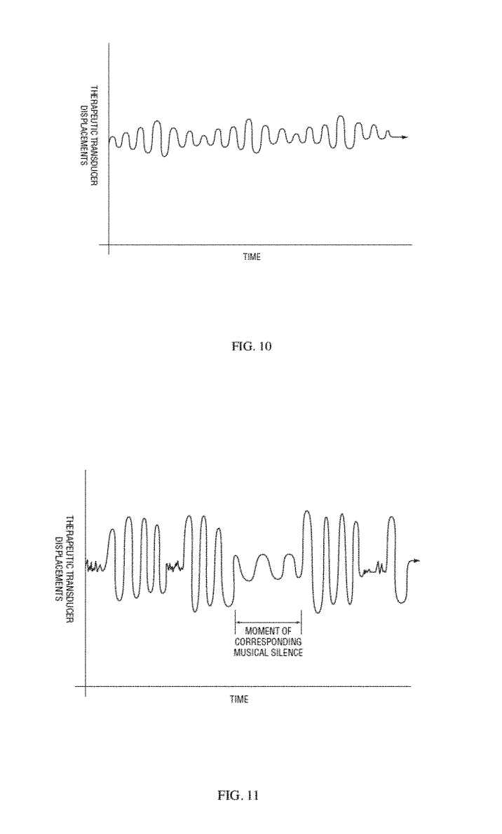

Processor 90 sorts the downloaded gaming and musical waveform, and in this case preferentially increases the amplitude of lower frequency bass tones (similar to as shown in FIG. 9) sent to and emitted by therapeutic transducer 10, which are generally closer to the resonance frequencies of the applied soft tissue, blood vessel and target internal organs.

A second application cord 41 feeds from processor 90 to a set of head phones 70 to concomitantly provide patient 20 with correlated in-synch listening pleasure from the down loaded, in this case unprocessed (or un-adulterated), MUSART gaming and musical track.

An arterial heat sensor 80 (advantageously an anemometer) is advantageously disposed adjacent therapeutic transducer 10 (i.e. with both elements oriented along the long axis of the radial artery) to enable confirmation that the tactile oscillation equipment is properly placed over the radial artery. A mini-processor (not shown) operable with the arterial heat sensor 80, automatically assesses for arterial heat and pulsatile flow, and provides illumination of a light 16 located on the face of pressure sleeve 50 to the color “green” when arterial heat and pulsatile flow is sensed, thereby confirmation the location of the target artery. Treatment session continues for about half an hour.

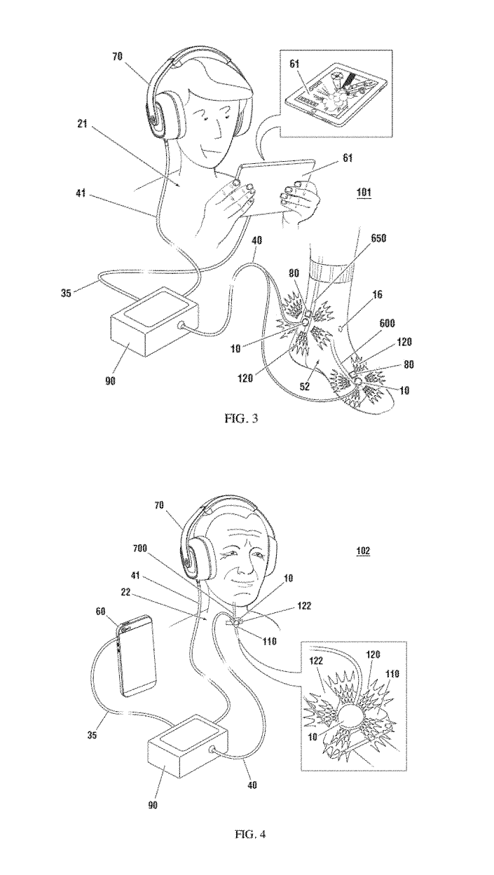

In Reference to FIG. 3, a perspective view of a patient 21 enjoying use of a MUSART therapy system 101 in a variation for treatment of a broken leg is shown.

A pair of therapeutic transducers 10 providing video game derived audible oscillations in the infrasonic to low audible frequency range (i.e. 1 Hz-20 KHz), are in this case secured and disposed by inflate-able pressure sock 52 for placement about the foot and ankle which is inflated and maintained automatically at 80 mm Hg, to provide localized, targeted placement and engagement force of therapeutic transducers 10 upon pedal artery 600 and posterior tibial artery 650 (shown as under the skin by dashed lines). Inflate-able elements of pressure sock 52 are not shown, being located on the bottom and lateral aspect of the foot, contra-lateral to their respecting areries. A video gaming sound track selected for treatment of a broken leg is down loaded to MUSART processor 90 from MUSART sound source (stored within the Internet—not shown) via use of IPAD 61 and processing cord 35, whereby this information is processed and fed from processor 90 to therapeutic transducers 10 via an application cord 40. Processor 90 sorts the downloaded video game audio waveforms to enable mechanical activation of therapeutic transducers 10 (with the resultant tactile vibration waves shown as a series of wave-fronts 120 emanating from the pedal and tibial artery application sites), and in this case processor 90 preferentially increases the amplitude of lower frequency bass tones and eliminates higher tones (i.e. above about 300 Hz) completely. In this case processor 90 also adds a 20 Hz base frequency sine wave amplitude modulator during times of audio expression (similar to as shown in FIG. 10), to further accentuate a more regular, internalized vibratory effect, at or near tissue resonance.

Application cord 41 from processor 90 feeds to head phones 70 to concomitantly provide patient 21 with correlated listening pleasure from the down loaded, in this case unprocessed (or un-adulterated), MUSART video gaming sound track. Patient 21 proceeds to play the downloaded video game, via IPAD 61.

An arterial heat sensor 80, again preferably an anemometer, is disposed alongside each therapeutic transducer 10 (with each pairing of elements strategically oriented along the length, or long axis of their respective artery), to enable confirmation, by monitoring of arterial heat and pulsatile flow that therapeutic transducer 10 is well placed upon each respective artery. A mini-processor (not shown) operable with arterial heat sensors 80 assesses for a temperature correlating to arterial emitted heat and provides illumination of light 16 disposed upon pressure sock 52 to the color “green” when adequate positioning of arterial heat sensors 80 (and thereby by inference, therapeutic transducers 10), over and upon the pedal and tibial artery are confirmed.

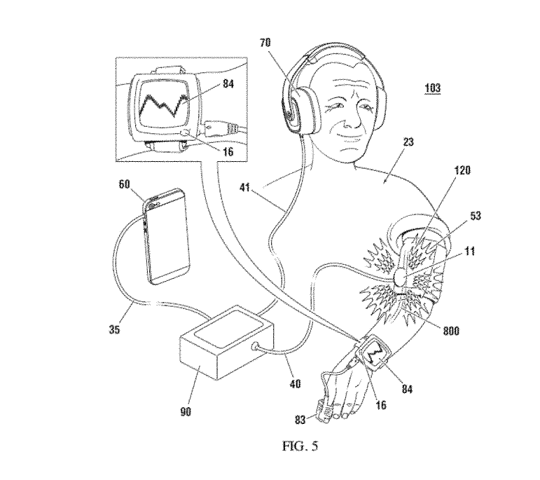

In Reference to FIG. 4, a perspective view of a patient 22 enjoying use of a MUSART therapy system 102 in a variation for rehabilitation treatment following a stroke with subsequent carotid arterial stenting is shown.

Therapeutic transducer 10 is in this embodiment acoustically attached to a generally triangular shaped applicator 110 (with slightly rounded corners) overlying carotid artery 700, and being sized to enable snug, fitted seating within the carotid triangle of the neck of patient 22, and having a dimensional width at the narrow base of the triangle (i.e. nearest the head of patient 22), of about 2.5 cm, which is greater than or equal to 1.5× the diameter of a typical carotid artery (to thereby ensure continued engagement of triangular shaped applicator 110 upon the carotid artery regardless of subtle movements or migration of applicator 110 during use). In this case therapeutic transducer 10 provides relaxing music derived tactile oscillations in the infrasonic to audible frequency range (i.e. 1 Hz-20 KHz), whereby a musical track selected for rehabilitation treatment of a stroke and enhanced carotid arterial healing is down loaded to MUSART processor 90 from MUSART sound source (stored within the Internet—not shown) via use of cell phone 60 and a processing cord 35, whereby this information is processed, and fed from processor 90 to therapeutic transducer 10 via an application cord 40.

Processor 90 sorts the downloaded musical waveforms to enable mechanical activation of the therapeutic transducer 10 (with vibration massage waves shown as a series of oscillatory wave-fronts 120 emanating from the carotid arterial application site), and in this case the amplitude of lower frequency bass tones in the 1 Hz to 300 Hz range are only slightly increased relative to the emitted higher audio tones sent to the therapeutic transducer (again, see FIG. 9), thereby promoting a relatively gentle and even therapy. Application cord 41 also feeds from processor 90 to head phones 70 to concomitantly provide patient 22 with correlated listening pleasure from the down loaded, in this case unprocessed (or un-adulterated), MUSART musical track.

Triangular shaped applicator 110 with therapeutic transducer 10 is expediently engaged to the skin surface of the neck of patient 22 by double sided adhesive tape 122 (although in a variation the underside, or skin facing side of applicator 110 may be made adhesive or sticky in nature, or a conventional tape may be disposed overlying applicator 110. In this embodiment, the weight of therapeutic transducer 10 along with applicator 110, (with patient 23 being advantageously reclined), enables sufficient engagement force against carotid artery 700 to enable sufficient tactile vibratory transmission. Alternatively, a small weight (not shown) may be applied atop therapeutic transducer 10 and triangular applicator 110, to add a further (e.g. 5 to 10 Newtons) of engagement force of therapeutic transducer 10 with triangular shaped applicator 110 upon the neck surface. Also, the pure musical tones for correlated listening (applicable by head phones and/or ear buds) will to a degree also send therapeutic oscillation waves across the temporal bone to the brain (and thereby non-specifically the cerebral arterial vasculature) for additional neurovascular angiogenic and blood flow stimulation effects. As the carotid artery is a relatively large and easy to locate artery (e.g. by palpation), heat sensor 80 as an arterial locator is not included as necessary in this embodiment, but, could be added as a variation.

It should be mentioned that while the above embodiment describes a very gentle vibratory tactile treatment of the brain and stented carotid artery post cerebral vasculature accident, the above assembly (or general equivalents, with an infrasonic to audible frequency vibrator non-invasively disposed on the carotid artery) may also be used for treatment of acute ischemic stroke—whereby hemodynamic pulsations applied to the carotid artery help flush, and thereby assist reperfusion of an acutely thrombosed cerebral artery.

The use of non-invasively imparted carotid arterial vibrations in the high infrasonic to low sonic frequency range for treatment of acute ischemic stroke (with a degree of serial compressions followed by decompressions of the carotid artery) has been well described in co-pending parent U.S. patent application Ser. No. 13/986,252 to Hoffmann et al, which is incorporated herein by reference. In this acute treatment variation (by use of MUSART therapy), the accentuating of bass tones, and addition of a base frequency amplitude modulator for the musically derived waveform with a base frequency signal applied during musical silence (similar to as seen in FIG. 10) is preferred to maximize tactile oscillations applied at or near vascular tissue resonance. It is also preferable, in this acute embodiment, to use a more powerful high fidelity vibration emitting motor (preferably a linear stepper motor, or rotary stepper motor with a cam, not shown) with higher displacement amplitude or stroke length enablement than therapeutic transducer 10, to maximize the potential for amplitude driven forces of vibration applied to the carotid artery, whereby vibration with a stroke length of up 0.1 mm to 1 mm or even 2 mm is preferred, depending on the size of the patient.

It should also be mentioned that direct stimulation of the temporal arteries (underlying the temple region) of the cerebral vasculature can also be achieved by direct acoustic stimulation, from a variation of therapeutic transducer 10 (comprising for example an acoustic transducer place able above and in-front of the ear of a user—either adhered directly to the skin surface, or applied by headphone, or in incorporated into the ear connectors (or extensions) of eye glasses. The temporal bone and jaw are excellent acoustic transmitters, hence local stimulation of the temporal arteries, alone, or in conjunction with carotid arterial stimulation (for the same listed purposes) are readily achievable in MUSART therapy according to the invention.

In Reference to FIG. 5, a perspective view of a patient 23 enjoying use of a MUSART therapy system 103 in a variation for providing pre-ischemic conditioning therapy to the heart prior to Coronary Artery Bypass Surgery (CABG) is shown.

A variant higher powered therapeutic transducer 11 providing tactile musically derived oscillations in the infrasonic to low audible frequency range (i.e. 1 Hz-300 Hz), is in this case secured and disposed by a pressure sleeve 53 which is automatically inflated by inflation elements (not shown) located on the contra lateral surface of the arm relative to brachial artery 800, and maintained to 80 mm Hg, to provide targeted placement and engagement force of variant higher powered therapeutic transducer 11 upon brachial artery 800. A musical track selected for cardiac ischemic pre-conditioning is down loaded to MUSART processor 90 from MUSART sound source (within an internet application—not shown) via use of cell phone 60 and a processing cord 35, whereby this information is processed with only the bass frequency range of the musical track (with higher frequencies of the musical piece filtered out by a low pass filter) being fed from processor 90 to variant higher powered therapeutic transducer 11 via an application cord 40. Mechanical activation of variant higher powered therapeutic transducer 11 yields correlated musical frequency vibration waves shown as a series of oscillatory wave-fronts 120 emanating from the brachial arterial application site.

Application cord 41 stemming from processor 90 and feeding head phones 70 concomitantly provide patient 23 with correlated listening pleasure from the down loaded, in this case unprocessed (or un-adulterated), MUSART musical track.

A finger plethysmograph 83, with display screen 84, is in this case advantageously disposed about the left finger tip and wrist respectively of patient 23, to enable confirmation, by monitoring, of musically derived phasic bass frequency wave distortions superimposed upon the otherwise naturally occurring finger tip arterial blood volume waveform trace (shown within display screen 84). Alternatively, an anemometer, a Doppler flow sensor (not shown) or a force sensor may also, or independently, be placed upon the carotid artery of patient 23, as a variant means for assessment of propagations of intra-arterial oscillation induced fluctuations in blood flow, velocity, or volume. A mini-processor (not shown) operable with finger plethysmograph 83 automatically assesses the degree of musically derived phasic distortions instilled upon the arterial blood volume trace, and provides illumination of a light 16 to the color “green” when adequate positioning and engagement force of the activated variant higher powered therapeutic transducer 11 over and upon brachial artery 800 is confirmed.

As the distance between the brachial arteries and the heart is quite substantial, variant higher powered therapeutic transducer 11 preferably contains a high powered, high fidelity linear stepper motor (capable of emitting relatively forceful, complex oscillatory wave shapes), to provide application of the bass frequency aspects of the selected musical audio waveform (i.e. at or below about 300 Hz) at a relatively increased displacement amplitude or stroke length—such as up to at least 1 mm and up to more preferably 2 mm or (for larger patients) 4 mm. This provides an extra strong vibratory response (seen again as the oscillatory wave-fronts 120 emanating from sleeve 53, at the position of the left brachial artery) to enable preferred significant compression and decompression of the brachial artery via the bass frequencies of the musical waveform. Processor 90 also inputs to variant higher powered therapeutic transducer 11 a base frequency sine wave amplitude modulator of 8 Hz (approximating a resonance frequency of the heart) which acts on the bass frequency musical carrier wave—whereby the 8 Hz sine wave is additionally applied during times of musical silence (similar to what is shown in FIG. 10). Treatment sessions for cardiac pre-ischemic conditioning generally last for about half an hour to an hour via the MUSART method.

It should be mentioned that the mechanism of how vibration provides cardiac ischemic—preconditioning is believed to involve increasing NO bioavailability within the epi-myocardium, as well as induction or priming recruitment of coronary collaterals, in anticipation of a possible ischemic insult. Cardiac pre-ischemic conditioning typically provides for a lasting effect, which remains for up to about 48 hrs, hence is a highly recommended pre-op therapy prior to CABG.

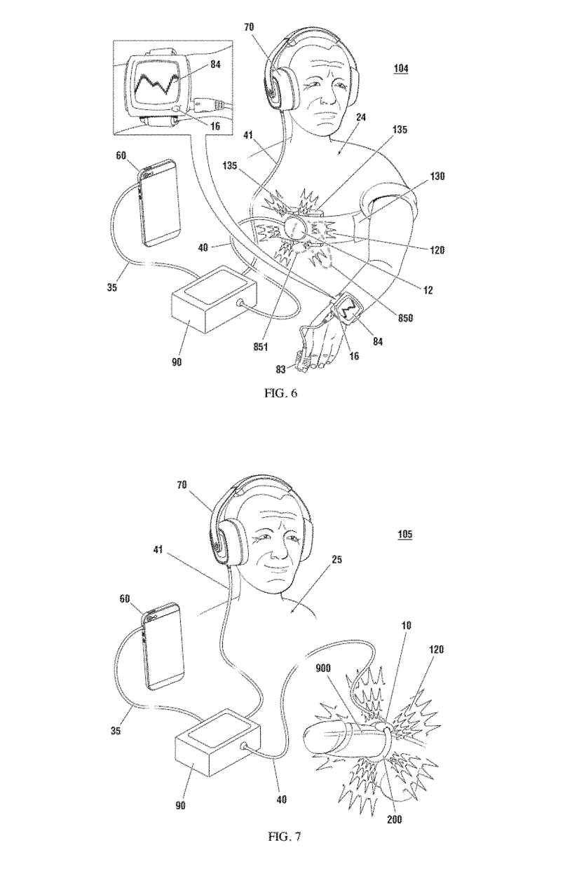

In reference to FIG. 6, a perspective view of a patient 24 enjoying use of a MUSART therapy system 104 in a variation for providing treatment for refractory angina (by induction of coronary angiogenesis) and enhanced coronary vessel healing following a coronary stent procedure to the left anterior descending artery (by promoting a decreased inflammatory response) is shown.

A variant high intensity transthoracic cardiac therapeutic transducer 12 providing tactile musically derived oscillations in the infrasonic to low audible frequency range (i.e. 1 Hz-300 Hz), is in this case disposed to the chest wall surface of patient 24 via a tight-enable elastic belt assembly 130 to provide targeted placement and engagement force of cardiac therapeutic transducer 12 via a pair of gel pad applicators 135 (disposed underlying elastic belt assembly 130) to the anatomic left and right of the sternum (at the level of the 3^{rd} and 4^{th} intercostals space) which anatomically conforms to the location of left coronary artery 850 and right coronary artery 851 (shown by dashed lines, within the thoracic cavity of patient 24). A musical sound track selected for cardiac angiogenesis and coronary healing is down loaded to MUSART processor 90 from MUSART sound source (stored within an internet application—not shown) via use of cell phone 60 and a processing cord 35, whereby this information is processed with only the bass frequency range of the musical track (with higher frequencies of the musical piece filtered out by a low pass filter) being fed from processor 90 to variant cardiac therapeutic transducer 12 via application cord 40. Mechanical activation of variant cardiac therapeutic transducer 12 yields vibration waves shown as a series of oscillatory wave-fronts 120 emanating from the chest wall application sites.

Application cord 41 feeds from processor 90 to head phones 70 to concomitantly provide patient 24 with correlated listening pleasure from the down loaded, in this case unprocessed (or un-adulterated), MUSART musical sound track.

Finger plethysmograph 83, with display screen 84, is in this case also preferably disposed about the left finger tip and wrist respectively of patient 24, to enable confirmation, by monitoring, of musically derived phasic bass frequency wave distortions superimposed upon the otherwise naturally occurring finger tip arterial blood volume waveform trace (shown within display screen 84). Alternatively, an anemometer, Doppler flow sensor or a force sensor may also, or independently, be placed upon the carotid or radial artery of patient 24, as a variant means for assessing intra-arterial oscillation induced fluctuations in blood flow, velocity or volume. A mini-processor (not shown) operable with finger plethysmograph 83 automatically assesses the degree of musically derived phasic distortions instilled upon the arterial blood volume trace, and provides illumination of a light 16 to the color “green” when adequate positioning and engagement force of the activated variant cardiac therapeutic transducer 12 over and upon the coronary arteries and heart is confirmed. In a lower tech solution, patient 24 may be asked to simply verbally recite in a low tone the word “ahhhhhhhh” whereby vibratory undulations in the vocal tone (heard by patient 24, or a third-party observer) also confirm transthoracic penetration of the vibratory waves to within the thoracic cavity (and thereby including the heart).

As the distance between the coronary arteries and the chest wall surface is quite significant (i.e. typically about 4 cm), variant cardiac therapeutic transducer 12 also preferably comprises a high fidelity linear stepper motor (which is advantageously capable of emitting complex oscillatory wave shapes), to provide application of the lower frequency bass frequency aspects of the selected musical audio waveform (i.e. at or below about 300 Hz) at a relatively high displacement amplitude or stroke length in the 0.1-10 mm range. Research by the applicant has shown that displacement amplitudes of at least 1 mm and up to more preferably 2 mm or (for larger patients) even 4 mm to 6 mm are required (with vibration emitted at lower frequencies) to achieve satisfactory transthoracic penetration. This provides an extra strong vibratory response (seen as oscillatory wave-fronts 120 emanating the chest wall application sites) to enable preferred penetration of vibration to the myocardium of the heart and coronary arteries via the bass frequencies of the musical waveform. Processor 90 also strategically inputs to cardiac therapeutic transducer 12 a base frequency sine wave amplitude modulator of 8 Hz (approximating a resonance frequency of the heart) which acts on the bass frequency musical carrier wave (similar to what is shown in FIG. 10), and whereby a 20 Hz to 120 Hz sinusoidal oscillation waveform (approximating the resonance frequency of the epi-myocardium), is additionally applied during times of musical silence (similar to what is shown in FIG. 11). Treatment sessions for treatment of refractory angina (by induced coronary angiogenesis) and enhanced coronary arterial vessel healing generally last for about half an hour to an hour per treatment session via the MUSART method.

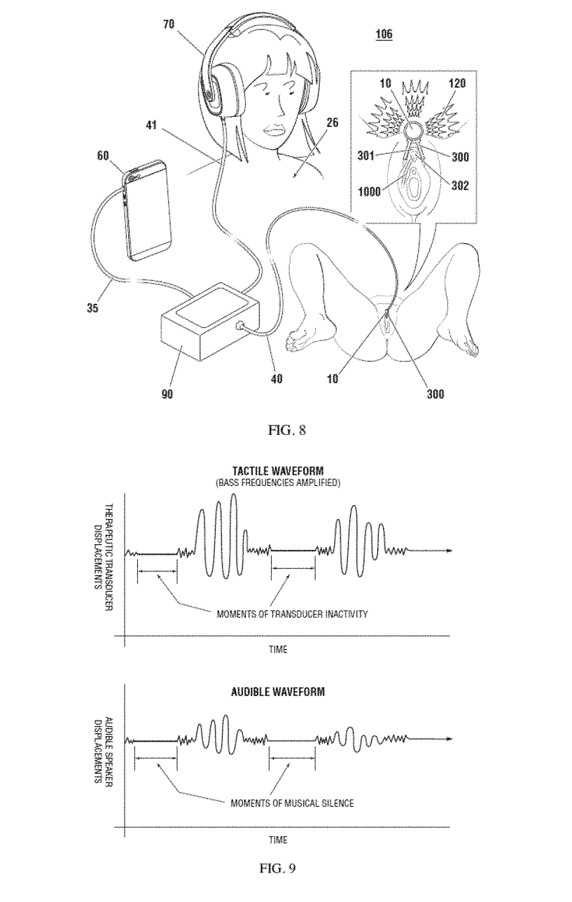

In Reference to FIG. 7, a perspective view of a male patient 25 enjoying use of a MUSART therapy system 105 in a preferred embodiment for treatment of erectile dysfunction and general enhancement of penis size is shown.

MUSART therapeutic transducer 10 is in this case disposed upon an elastic ring 200 sized for placement over an erect, or partially erect penile shaft (whereby the elastic ring 200 doubles as both an engagement means and an applicator for therapeutic transducer 10) whereby therapeutic transducer 10 is oriented superiorly (with respect to erect penis 250) to seat upon the dorsal artery 900 (shown as under the skin by dashed lines). A musical sound track selected for enhanced NO bioavailability to the penis and treatment of erectile dysfunction is downloaded to a MUSART processor 90 from a MUSART sound source (stored within an internet application—not shown) via use of cell phone 60 and inter-connecting processing cord 35, whereby this information is processed, and fed from processor 90 to therapeutic transducer 10 via an application cord 40, to emit musically derived vibratory waves (shown as a series of oscillatory wave-fronts 120) emanating from elastic ring 200, at the superior aspect of the erect penis at the position of dorsal artery 900 target application site.

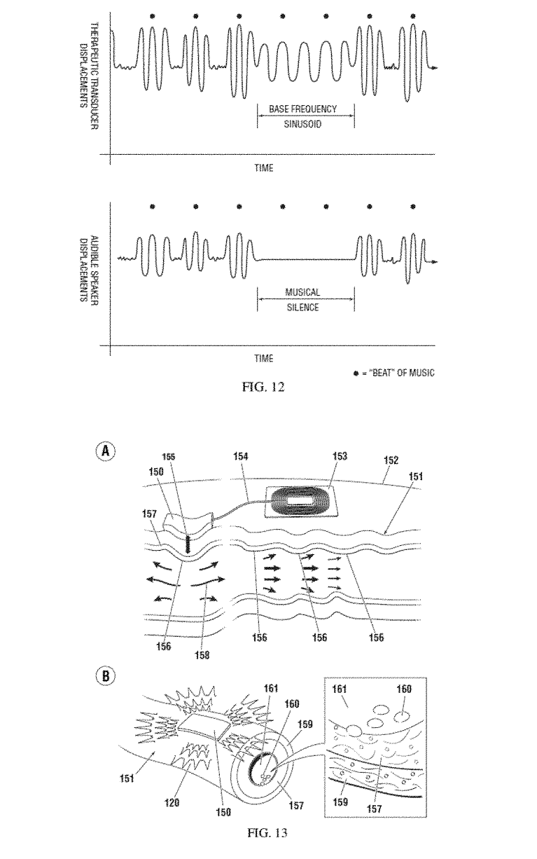

Processor 90 sorts the downloaded musical waveform, and in this case preferentially increases the amplitude of lower frequency bass tones sent to and emitted by therapeutic transducer 10 (similar to as shown in FIG. 9), which are generally closer to the resonance frequencies of the applied soft tissue, blood vessel and target internal organs. Base frequency amplitude modulation via a sinusoidal wave (like as shown in FIG. 10) is also advantageously blended with the tactile musical “carrier” wave, and is also (more advantageously) periodically emitted as a pure sinusoidal tone during periods of musical silence (like as shown in FIG. 11). Most advantageously, the base frequency sine wave is blended in synch with, and has a frequency which either matches or comprises a harmonic multiple with respect to the cadence or rhythm (or beat frequency) of the musically derived inputted waveform (i.e. in other words the base frequency sine wave has amplitude peaks which are in synch with and are timed to match, or comprise a harmonic multiple to the cadence or rhythm of the musically derived inputted waveform—like as shown in FIG. 12) to add to the enjoyment of the combined tactile with correlated listening experience.

Application cord 41 feeds from processor 90 to head phones 70 to concomitantly provide patient 25 with correlated in-synch listening pleasure from the down loaded, in this case unprocessed (or un-adulterated), MUSART musical track.

Plastic ring 200 is optionally made inflate-able (an inflate-able variant is not shown) to provide targeted placement and diastolic engagement force (in the range of 40 mm Hg to 100 mmHg, nominally 80 mm Hg) of therapeutic transducer 10 against penile arteries—which as stated earlier optimally provides systolic flow and to a safe degree limits venous flow (for increased penis size), while further enhancing penetration of vibration from therapeutic transducer 10 to the penile arteries for optimized vascular NO-dependent and enhanced vasodilation or enhanced circulation effects. As described earlier the application of transducer 10 to the superior aspect of the erect penis is advantageous, as this configuration potentially can lead to stimulation of a female clitoris and G spot during intercourse, whereby a female partner may optionally (via an extra set of head phones) enjoy MUSART therapy co-jointly with her male partner, who is wearing the apparatus.

In Reference to FIG. 8, a perspective view of a female patient 26 enjoying use of a MUSART therapy system 106 applied to a clitoral artery 1000, with therapeutic transducer 10 in this case placed by a clip 300 overlying the clitoral hood 301 being in intimate contact with clitoris 302, with a musical track selected for inducing enhanced blood flow and increased NO bioavailability to the clitoris according to the invention is shown.

MUSART therapeutic transducer 10 is disposed upon clip 300 being sized for placement upon the clitoral hood 301 (whereby clip 300 doubles as both an engagement means and an acoustic transmission applicator for therapeutic transducer 10), whereby therapeutic transducer 10 provides tactile vibrations to clitoris 302 and clitoral artery 1000. A musical track selected for enhanced NO bioavailability to the female genitalia is downloaded to a MUSART processor 90 from a MUSART internet application via use of cell phone 60 and inter-connecting processing cord 35, whereby this information is processed, and fed from processor 90 to therapeutic transducer 10 via an application cord 40, to emit musically derived vibratory waves (shown as a series of oscillatory wave-fronts 120 emanating from clip 300).

Processor 90 sorts the downloaded musical waveform, and in this case preferentially increases the amplitude of lower frequency bass tones sent to and emitted by therapeutic transducer 10 (similar to as shown in FIG. 9), which are generally closer to the resonance frequencies of the applied soft tissue, blood vessel and target internal organs. Base frequency amplitude modulation via a sinusoidal wave (like as shown in FIG. 10) is also advantageously blended with the tactile musical “carrier” wave and is also (more advantageously) also emitted as a pure sinusoidal tone during periods of musical silence (like as shown in FIG. 11). Most advantageously, the base frequency sine wave is blended in synch with and either matches or comprises a harmonic multiple with respect to the cadence or rhythm of the musical piece derived from the musically derived inputted waveform (like as shown in FIG. 12) which greatly harmonizes and accentuates tactile with correlated listening pleasure. The application of transducer 10 to the superior aspect of the female genitalia (proximate the clitoris) is additionally advantageous, as this configuration potentially can lead to stimulation of a male penis during intercourse, whereby a male partner may optionally (via an extra set of head phones) enjoy MUSART therapy co-jointly with his female partner, who is wearing the apparatus. Also, in a variation for stimulation of the external female genitalia, therapeutic transducer 10 may be applied singularly to the clitoral hood or clitoris directly (e.g. by use of an adhesive), or a plurality of therapeutic transducers 10 may be applied to both the clitoris and clitoral hood simultaneously to enable multiple sites of acoustic stimulation to the clitoris and clitoral arteries (including the deep and dorsal clitoral arteries). Moreover, it is also conceivable that therapeutic transducer 10 (or a larger variant) may be disposed within a female undergarment (e.g. panties) to provide required clitoral stimulation.

Application cord 41 feeds from processor 90 to head phones 70 to concomitantly provide patient 26 with correlated in-synch listening pleasure from the down loaded, in this case unprocessed (or un-adulterated), MUSART musical sound track.

In reference to FIG. 9 a pair of graphs (with displacement amplitude representing the vertical axis, and time representing the horizontal axis) of; (top) an emitted musically derived tactile waveform whereby the relatively lower frequency bass tones have been amplitude enriched relative to the higher frequency tones, versus (bottom) its correlating, in-synch audible waveform, or (“musical piece”) for correlated listening which shows relatively lower bass frequency amplitude relative to the higher frequency tones, is shown. In this example (in reference to the top graph) the lower frequency musically derived “bass” tones (i.e. with waveform frequencies in the 1-300 Hz range) have been amplitude enriched relative to the higher frequency tones as emitted by a therapeutic transducer, as compared to (in reference to the bottom graph) the audible waveform emitted by an audible speaker. In this example, no sinusoidal base frequency amplitude modulation or transiently emitted sinusoidal tones (i.e. during moments of transducer inactivity or “musical silence”) have been added to the provided tactile oscillation massage waveform. Straight lines on the graph, free of undulations (top) refer to periods of no MUSART therapeutic transducer activity (i.e. no tactile oscillations emitted), and (bottom) refer to correlated periods or moments of musical silence (i.e. no audible oscillations emitted) with respect to FIG. 9.

The accentuation of the lower frequency bass musical tones of an audio derived waveform, less than about 300 Hz, and preferably at least 8 Hz, and commonly in about the 20 Hz to 120 Hz range, is generally preferred as such lower tones can be safely and comfortably applied to the human body at relatively higher displacement amplitudes or intensities (or stroke lengths), are at a high enough frequency to produce vascular turbulence and sheer producing intravascular effects, and are generally in the range of frequencies co-incident with at least one of; tissue, arterial and/or target organ resonance within the human body (which thereby serves to optimize the produced internal and transmitted therapeutic vibratory effect).

In reference to FIG. 10 a graph view of an emitted musically derived oscillatory massage waveform, with again oscillatory displacements (which correlates to the degree of oscillatory movement, or displacement amplitude, of the oscillating engagement face or applicator of the utilized tactile therapeutic transducer) representing the vertical axis, and time representing the horizontal axis, is shown. In this example, a base frequency sine wave amplitude modulator has been added to a mono-amplitude exclusively bass frequency musically derived oscillatory massage waveform, which may be described, in relation to the base frequency modulator, as the carrier wave. Addition of a base frequency sinusoidal amplitude modulator of the musically derived bass frequency mechanical oscillation signal (for example in the range of about 8 Hz to 300 Hz, and preferably in most cases 20 Hz-120 Hz, and most preferably harmoniously coordinated and in-synch with the beat or cadence of the musical waveform or piece) is advantageous, to further ensure a strong time weighted average of mechanical oscillations emanating from the utilized MUSART therapeutic transducer are available for demodulation within the human body at or near tissue, or vascular sheer producing resonance.

In reference to FIG. 11 a graph view of an emitted musically derived oscillatory massage waveform, with again oscillatory displacement amplitude (which correlates to the degree of oscillatory movement, or displacement amplitude of the oscillating engagement face or applicator of the utilized therapeutic transducer as applied to the user's body surface) representing the vertical axis, and time representing the horizontal axis, is shown. In this example, an emitted base frequency sine wave signal applicable during a “moment of musical silence” has been strategically added to the musically derived tactile oscillatory massage waveform—whereby in this case the “bass” frequency range is additionally amplitude enriched relative to the higher pitched frequencies (similar to as shown in FIG. 9).

Addition of a tactile sinusoidal oscillation wave during moments of correlated musical silence (for example in the range of about 8 Hz to 300 Hz, and preferably in most cases 20 Hz-120 Hz, and most preferably harmoniously coordinated with the frequency of amplitude peaks in-synch with the beat frequency or cadence of the musical waveform or piece—like as shown in FIG. 12) is advantageous, to further ensure a strong time weighted average of mechanical oscillations emanating from the selected MUSART therapeutic transducer are available for demodulation within the human body at or near tissue or vascular sheer producing resonance. The regularly appearing sine wave drawn on the graph of FIG. 11 corresponds to the emission of a tactile base frequency sinusoidal oscillation wave during what is labeled as a “moment of musical silence”, with respect to the audible emissions utilized for correlated listening.

In reference to FIG. 12, a graph view (with displacement amplitude representing the vertical axis, and time representing the horizontal axis) of; (top) an emitted musically derived oscillatory waveform with an added base sinusoidal waveform emitted during a time of correlated musical silence—for tactile massage—; versus (bottom) its correlating, in-synch audible oscillatory waveform (or “musical piece) which shows a temporally matching frequency and wave-shape—for correlated listening—is shown. Note how the distance between peaks of the tactile sinusoidal waveform are synchronized as a multiple (in this case double) to the rhythm or beat frequency (beats shown as dots on the graph) of the musical piece for added harmonious tactile and listening pleasure.

Many modifications are possible with regards to the MUSART system, without departing from the spirit or innovative concept of the invention

For example, while the preferred embodiment shows use of musical sound tracks and/or alternatively video gaming technology to enable tactile massage with correlated listening, alternatively, it is conceivable that movie tracks (particularly musicals), or even television show tracks (preferably with music, or lots of exciting sounds like explosions) may optionally be employed according to the invention.

Also, while the preferred embodiment shows only use of one therapeutic transducer applied to (essentially) a single application site upon a single artery or arterial network of a user, alternatively a plurality of therapeutic transducers may be employed either along or upon one target artery, or upon at least two, and perhaps several target, palpable arteries, to further ensure engagement and transmission of both local and systemic vascular effects to a user's blood stream.

Also, while the preferred embodiment shows use of a “speaker” with respect to MUSART therapeutic transducer 10 (for gentle, low amplitude applications) and a linear stepper motor with respect to the variant, higher intensity transducers (for more forceful, higher displacement amplitude—or stroke length—applications), alternatively any number of alternative oscillatory motors which enable emission of reasonable high fidelity, variable oscillation waveforms—such as (but not limited to)—eccentric spinning weights, a rotary motor, a rotary stepper motor with a cam, a linear motor—may be incorporated as variations according to the invention.

Also, while the preferred embodiment shows a range of engagement means for therapeutic transducer 10, most commonly comprising inflate-able sleeves which enable engagement forces of therapeutic transducer 10 against a body surface at or around a diastolic pressure of a user-alternatively many other engagement means may be considered. For example, therapeutic transducer 10 may be placed within a condom (also optionally inflate-able), whereby the condom disposes therapeutic transducer 10 to the shaft of the penis for stimulation of the penile arteries. For example, engagement of therapeutic transducer 10 to the pedal artery or radial artery may be accomplished by a sock or glove respectively, or alternatively a bracelet, (all of which being optionally inflate-able). For example, engagement of therapeutic transducer 10 to the carotid artery of a user may be accomplished by a neck band or snugly fitting necklace, or neck tie. Essentially any means of positioning therapeutic transducer 10 to a select body surface, with some degree of set, or controllable engagement force (to keep therapeutic transducer stably positioned) may be used, according to the invention.

Moreover, an acoustic tactile sound source (or arterial parameter detector, e.g. blood flow sensor) enabling MVT therapy targeted to an artery may be adhered directly to the skin surface, conceivably by ultra thin electronics. Ultra-thin electronics adhered directly to a client's skin surface for physiological applications (termed “electronic tattoos” or “epidermal electronics”) is a growing, and now commercially available field in modern medicine and health related concerns. Taking advantage of recent advances in flexible electronics, it is recently possible to “print” devices directly onto the skin so people can wear them for an extended period (typically about 2 weeks at a time) while performing normal daily activities. Such electronics can be delivered preferably by use of a rubber stamp or can be more carefully implemented (such as by the equivalent of a tattoo artist)—whereby the ultrathin mesh electronics are applied directly to the surface of the skin overlying a target artery. Further, one can additionally utilize a commercially available “spray-on bandage” product to add a thin protective layer and bond the system to the skin in a very robust way. Epidermal electronic devices may be powered wirelessly by an external power source and may receive and emit information by telemetry. Any of the embodiments in use of therapeutic transducer 10 or variations thereof, may alternatively employ electronic tattoo technology (e.g. where an arterial sensor or acoustic transducer for example may be “printed” or equivalently adhered along the course of an artery). Of note, ultra thin acoustic transducers would enable emission of a very gentle form of tactile vibration, which may have preferred use therefore invasively, with the ultrathin acoustic transducer for example applied directly upon the outer surface of a target artery (discussed more in-detail below).

Also, while the preferred embodiment shows a non-invasive positioning of a MUSART therapeutic transducer proximate or overlying a target artery, it is also conceived in the present invention that a MUSART therapeutic transducer may be placed invasively, within the body of a user—generally proximate and in acoustic contact with a target artery. For example, a MUSART therapeutic transducer may be placed in the pectoral pocket proximate a subclavian artery of a user (similar to pacemaker insertion sites) or may be placed under the skin posterior the scrotum for stimulation of perennial arteries. In this invasive embodiment, the massaging oscillation waves may or may not be necessarily “tactile” in that the vibrations may or may not be actually felt by a user, however penetration to the target artery is often superior (vs. non-invasive applications) as the MUSART therapeutic transducer can be brought in very close proximity, or even touching the target artery. In reference to FIG. 13(a), a MUSART thin, flexible therapeutic transducer 150 is shown implanted proximate (and in this adhered, or “printed” upon) a target artery 151, under the skin 152. An electromagnetic charging coil 153 is disposed proximate and in connection (by connector 154) with flexible therapeutic transducer 150, to enable non-invasive wireless recharging of the invasive system. Therapeutic transducer oscillations 155 yields vibrations 156 of endothelial layer 157 with production of intraluminal force vectors 158 (which yield therapeutic endothelial sheer stress) which generally depicts the therapeutic action of vascular MUSART therapy. In reference to FIG. 13 (b) artery 151 is again shown with flexible therapeutic transducer 150 disposed on its outer surface (with oscillatory wave-fronts 120 emanating from transducer 150). Artery 151 is shown in cross section with smooth muscle layer 159 surrounding endothelial layer 157 being stimulated by oscillatory wave-fronts 120 to thereby liberate endothelial derived beneficial molecular mediators 160 to vessel lumen 161 which transmit and biosignal therapeutic circulatory effects.

As discussed above, use of ultrathin, flexible electronics (including ultrathin/flexible sensors and acoustic transducers) enable positioning of an acoustic sound source (such as in MVT therapy) directly upon, and/or encircling a selected target artery (such as to preferably run for a length along the artery) to enable direct invasive acoustic stimulation of the target artery. Such a technique could for example be utilized in Coronary Artery Bypass Surgery—whereby a ultrathin flexible electronic sleeve comprising a acoustic transducer +/− blood flow sensors may be disposed along the length of a native coronary vessel or bypass graft. Periodic acoustic stimulation of the coronary vessels or bypass grafts (whereby the acoustic waveform information, such as musical waves, could be sent by telemetry and bypass grafts would keep them healthy and free of clot.

Also, while the preferred embodiment shows a MUSART sound source received via Internet by a phone, gaming platform, tablet or other network connected electronic device, whereby the information is thereby transferred to a separate MUSART processor, whereby the information is thereby transferred to headphone speakers and to a separate MUSART therapeutic transducer, alternatively the MUSART SYSTEM may be contained within a single unit (e.g. smart phone, tablet, gaming system etc.—e.g. where the cell phone is equipped with a MUSART therapeutic transducer). Moreover, while the embodiments shown utilize wires to connect the components of the MUSART system, alternatively the system could be made wireless (whereby information is transmitted by telemetry etc.).

Also, while the embodiments shown demonstrate MUSART therapy primarily for use in treatment of pathologic, injury or recovery type conditions, alternatively the positive blood flow stimulation effects (including vascular angiogenesis and increased NO bioavailability) may also be used to bolster a user's immune system, or athletic and cognitive performance. For example, MUSART therapy applied to the legs would stimulate angiogenesis to the leg muscles, which would improve performance in sporting events involving running, kicking, cycling, or jumping. MUSART therapy applied to the carotid or temporal arteries would enhance cerebral vasculature angiogenesis, NO bioavailability and blood flow to the brain, which would help concentration, mental acuity, and mental aptitude. MUSART may also of course be employed with animals in veterinarian or live stock enhancement pursuits (e.g. better health, superior growth).

Finally, while the embodiments shown demonstrate mentally stimulating and cognitively meaningful musical and/or gaming sounds to effect MUSART therapy, it is also conceived that recitations of select Biblical text (particularly when delivered in the Hebrew language—which is known to be a sacred language) may also comprise an effective soundtrack for healing and well being, particularly for those who have faith. Moreover, recitations from the Quran (particularly in Arabic) are also believed by many to carry a divinely inspired melodic tone, which may carry additional healing powers, and hence may also be useful as a sound source in implementation of MUSART therapy.

### SUMMARY OF THE INVENTION

A method for imparting targeted vibration massage directly to an arterial vasculature of an individual with correlated listening including but not limited to the steps of;
a) providing a mechanical oscillation transducer enabling targeted, localized placement generally proximate a target artery,
b) providing a waveform signal input derived from a mentally stimulating or cognitively meaningful sound track downloadable to the transducer, the transducer enabling emission of a comparative tactile oscillation waveform having a matching frequency and wave-shape to the waveform signal input,
c) locating the target artery,
d) positioning the transducer to an application site proximate the target artery to enable transmission of the tactile oscillation waveform from the transducer to the target artery,
e) applying the tactile oscillation waveform via the transducer to acoustically stimulate the target artery
f) providing means for listening to an audible waveform derived from the mentally stimulating sound track, the audible waveform being temporally synchronized and having a matching frequency and wave-shape with respect to the tactile oscillation waveform, wherein the tactile oscillation waveform oscillates the target artery to provide a beneficial blood flow response to the individual, while the audible waveform is simultaneously listened to by the individual to provide a correlative harmonized and pleasurable listening experience to the individual.

A method as above described, whereby the transducer is positioned non-invasively, generally overlying the target artery.

A method as above described, whereby the locating of the target artery is accomplished by palpation.

A method as above described, wherein the locating the target artery is accomplished by at least one of; a heat sensor, an anemometer, a doppler flow sensor, and an accelerometer—disposed alongside the transducer.

A method as above described, wherein the transducer emits oscillations with a waveform frequency in the 1 Hz to 20,000 Hz range.

A method as above described, wherein the transducer emits vibrations with a waveform frequency in the 8 Hz to 300 Hz range.

A method as above described, wherein a bass frequency of the tactile oscillation waveform is amplified relative to higher pitched frequencies.

A method as above described, whereby the tactile oscillation waveform is amplitude modulated.

A method as above described, wherein the tactile oscillation waveform is amplitude modulated by a base waveform having a frequency lying in a range comparable to a resonance frequency of at least one of a tissue, organ and vascular target of said transducer.

A method as above described, wherein the base waveform is a sine wave.

A method as above described, wherein the tactile oscillation waveform is combined with a base oscillation waveform with a waveform frequency in the 1-300 Hz range during moments of corresponding inactivity or silence with respect to emission of the audible waveform.

A method as above described, wherein the base oscillation waveform has amplitude peaks temporally synchronized with a cadence or rhythm of a musical piece derived from the audible waveform.

A method as above described, wherein the transducer enables emission of a displacement amplitude of vibration of at least 1 mm, thereby promoting penetration of the vibration.

A method as above described, wherein said transducer is fastened to a body surface generally proximate the target artery by at least one of; a sleeve, a ring, a band, a bracelet, a sock, a glove, a belt, a clip, a condom—disposed about a body part of the individual, or by use of a sleeve inflatable up to a pressure coincident with a physiologic, or life supporting arterial pressure of the individual.

A method as above described, wherein the transducer is attached to a body surface generally proximate the target artery via an adhesive.

A method as described above, wherein the mentally stimulating sound track consists of a musical sound track, and the audible waveform is music.

A method as described above, wherein the mentally stimulating sound track consists of at least one of a video game sound track, television show sound track and movie sound track.

A method as above described, further having the step of maintaining a positioning of the transducer upon the target artery by use of a biofeedback sensor consisting of at least one of; an arterial heat sensor, an anemometer, an accelerometer and a Doppler flow probe, wherein said biofeedback sensor is disposed alongside the transducer in an orientation enabling co-incident disposition of the transducer and the biofeedback sensor along the long axis of the target artery.

A method as above described, further having the step of ensuring the target artery is being oscillated by the transducer by use of a biofeedback sensor disposed non-invasively upon a palpable artery remote from the application site of the transducer overlying the target artery, whereby the sensor measures propagating hemodynamic fluctuations arising from the target artery and reaching the palpable artery during activation of the transducer.

A method as above described, wherein the sensor is at least one of a plethysmograph, anemometer, accelerometer, and a doppler flow probe.

A method as above described, whereby said transducer is at least one of a:

speaker, an eccentric spinning weight, a rotary motor, a rotary stepper motor with a cam, a linear motor, and a linear stepper motor.

A method as above described, whereby the waveform signal of the mentally stimulating sound track is downloadable for use from a social media network.

A method as above described, whereby the transducer has at least one of an oscillatory engagement face and an applicator engagement face with a surface dimension at least 1.5 times a diameter of the target artery, to ensure maintained acoustic contact of the active end of the transducer with the target artery.

A method as above described, for use in at least one member of the group consisting of; treatment of arthritis, wound healing, healing of broken bones, improving mental acuity, improving athletic performance, promoting cerebral vasculature angiogenesis, healing of carotid artery, prevention of in-stent re-stenosis, prevention of in-stent thrombosis, pre-ischemic conditioning for the heart, pre-ischemic conditioning for the brain, treatment of acute arterial thrombosis, treatment of heart attack, treatment of acute ischemic stroke, treatment of refractory angina, promotion of coronary angiogenesis, treatment of erectile dysfunction, growth of penis size, intercourse, clitoral stimulation, and enhanced sexual experience.

A method for stimulation of the external genitalia of an individual for improved sexual experience, consisting the steps of;
a) providing an oscillation transducer enabling targeted, localized placement upon an external genital organ generally proximate a target artery supplying said genital organ,
b) providing a waveform signal input derived from a mentally stimulating sound track downloadable to said oscillation transducer, said oscillation transducer enabling emission of a comparative tactile oscillation waveform having a matching frequency and wave-shape to said waveform signal input,
c) providing means for said individual to listen to an audible waveform derived from said mentally stimulating sound track, said audible waveform being temporally synchronized and having a matching frequency and wave-shape with respect to said tactile oscillation waveform,
d) locating said genital organ and fastening said transducer upon said external genital organ proximate said target artery,
e) applying said tactile oscillation waveform to said external genital organ via said transducer which thereby stimulates said target artery, and
f) activating said means for said individual to listen to said audible waveform during said applying said tactile oscillation waveform prior to termination of said applying said tactile oscillation waveform, whereby said tactile oscillation waveform oscillates said target artery to provide a beneficial blood flow response and erotic sensations to said genital organ, while said audible waveform is simultaneously listened to by said individual to provide a correlative harmonized and pleasurable listening experience to said individual.

A method as above described, wherein the genital consists of at least one of the penis, clitoris and clitoral hood.

A method as above described, wherein the tactile oscillation transducer is fastened to the penis by at least one of a ring and a sleeve disposed about the shaft of the penis.

A method as above described, wherein the tactile oscillation transducer is fastened to said at least one of the clitoris and hood of the clitoris via at least one of a clip and an adhesive and in males where the transducer is disposed proximate the scrotum.

A method as above described, wherein the mentally stimulating sound track comprises a musical piece, and said audible waveform comprises music.

A method as above described, wherein the tactile oscillation waveform is complimented with a second, distinct oscillation waveform being emitted from the therapeutic transducer during moments of in-activity of the tactile oscillation waveform.

A method as above described, wherein the second oscillation waveform is sinusoidal waveform, with amplitude peaks temporarily synchronized with a beat frequency or tempo of a musical piece derived from the audible waveform, and wherein the tactile oscillation waveform is amplitude modulated.

An invasive method for acoustically stimulating an artery, comprising the steps of: a) providing a mechanical oscillation transducer enabling targeted, localized placement generally proximate a target artery, b) providing a waveform signal input derived from a mentally stimulating or cognitively meaningful sound track downloadable or streamed to the transducer, said transducer enabling emission of a comparative oscillation waveform having a matching frequency and wave-shape to said waveform signal input, c) locating said target artery, d) positioning said transducer proximate and in acoustic contact with said target artery to enable transmission of said tactile oscillation waveform from said transducer to said target artery, e) applying said tactile oscillation waveform via said transducer to acoustically stimulate said target artery f) providing means for listening to an audible waveform derived from said mentally stimulating sound track, said audible waveform being temporally synchronized and having a matching frequency and wave-shape with respect to said oscillation waveform, wherein said oscillation waveform oscillates said target artery to provide a beneficial blood flow response to said individual, while said audible waveform is simultaneously listened to by said individual to provide a correlative harmonized and pleasurable listening experience to said individual.

A method as above described, wherein the transducer is re-charged by an electromagnetic coil, wireless charging system, and/or whereby the transducer is adhered upon an outer surface of said target artery.

### Example 1: Living Organism can Respond to Text Cryptographically Converted to Music

We have discovered that religious text encrypted to form musical notes can modulate activity in biological systems. Encryption systems are known that use algorithmic text and music method modulating biological systems using the range of approaches, and creating a unified interface for text, music, computer science, computational and cellular-subcellular integration. Approaches that can be used are both within and beyond musical practice including but not limited to ALife, cybernetics, systems art, AI models and algorithms, evolutionary algorithms, agent-based modelling and self-organizing systems, adaptive behavior and interactive performance systems, and ecosystemic approaches to composition and computational effects on molecular pathways, biological autonomy and agency, and the poetics of biologically-active algorithms.

We have discovered that we can harness behaviors inspired by natural systems, formalized by biologists and computer scientists into algorithms, in order to develop, perform, affecting biological function by applying vibroacoustic instruments to vascular interface.

One unexpected form of transmitting messages to biological systems is through music. Its known as music cryptography, which is a method in which the musical notes A through G are synced or desynced with words or textual codes (such as Jewish Gematria) forming vibroacoustic effector-pattern. Using Musical Cryptography, it is possible to integrate customized designed messages inside melodies by mapping notes to letters and in order to control cellular function

We have shown that these cryptographic messages can be deciphered by mechanosensor cellular protein system independent of high brain function using biophysical and biochemical specific responses to the various sharps and flats in the musical keys connected to the textual message.

We have discovered that music cryptography presents novel ways of transporting meaningful or incomprehensible messages on cellular and subcellular levels in biological systems.

The Harmonic Alphabet also presents method of using synthetic bio-cryptographic messages without actual words, in which convey code capable to induce and modulate biological processes. Written letters don't have this advantage. We think that they can also be used to create randomized frequency patterns that are “understood” via cellular and subcellular mechanisms.

One such encryption software is the Solfa Cipher that turns text into singable melodies. Rather than use 26 different pitches for each letter of the alphabet, Solfa Cipher maps letters onto only seven notes of a musical scale (Do, Re, Mi, Fa, Sol, La, Ti) combined with unique durations. Solfa Cipher can be accessed for free at http://www.wmich.edu/mus-theo/solfa-cipher/, among other places.

There are no known examples of music encryption being used to influence biological processes. Systems thinking can be applied to biological systems at a range of levels: a cell is a system of chemical and energy transfer; the heart is an open oscillatory system; an organism is a self-regulating system that operates to keep certain critical parameters within acceptable boundaries to prevent death as the mathematical point attractor.

The language, trajectory, transition rules, attractors, basins of attraction, etc. intrinsically and algorithmically connected to sonic and musical effects on biological rhythmic perception using oscillatory molecular mechanisms and mathematical models. Nevertheless, in biological systems certain mathematical rule sets represented by an astonishingly complex range of global dynamics emerge from simple, local interactions.

Furthermore, the use of sonically situated adaptive processes can be seen as a structural coupling of a cell and environment. This is a critical construct in autopoietic theory used to describe a process of engagement which effects a sequence and order of recurrent interactions leading to the structural congruence between two (or more) biological systems. It can lead to the emergence of cooperative as well as competitive survival strategies on subcellular and super cellular levels, increasing the molecular behavioral diversity.

Weaving together equations from complex systems, biology, cell behavior and so on will allow creation of new modalities that employ biological diversity, rather than merely representing nature.

Text and Music (especially religious) Encoded jointly using various techniques do not merely represent a range of frequency but rather a bio-encryption key that is defined by four elements: a clef, a tonic, a mode, and a rhythmic unit. When a religious text-musical cipher encodes a highly complex message, decrypting using intellectual capacity of the music theorists and cryptographers is not possible. Nevertheless, such text and music holds intricate patterns and biological meaning that are beyond conscious deciphering and can directly influence function of biological systems.

Methods of musically encrypting text include but are not limited to: ALife, artificial chemistry, connectionism, artificial intelligence (AI) models and algorithm, cybernetics, cryptography, systems art, AI s, evolutionary algorithms, agent-based modelling, self-organizing systems, adaptive behavior, interactive performance systems, ecosystemic approaches to composition and internet of things (IoT), computational effects on molecular pathways, biological autonomy biological agency, poetics of biologically-active algorithms, optimization and problem-solving, autonomy, self-determination, algorithmic music, Multi-Agent Compositions, interactive software systems, electro-acoustic partnerships, general systems theory (GST), general principles of dynamic interaction, regulatory feedback, Mandelbrot Sets, Lorenz Attractors principals, Mel-Frequency Cepstral Coefficients (MFCCs), system aesthetics, behavioral tendency, artificial neural networks, biological coupling, cellular automata, swarming, metaheuristic optimization, genetic algorithms, MutaSynth, Synthbot, SolfaCipher, PatchMutator, Biomorphs, Filterscape, Frequency Modulation (FM), non-linear (exponential, logarithmic or cubic) mappings, nested sub-functions mutations, distributed interactive genetic algorithm (IGA), artificial critics, beat tracking, principles of situatedness and embodiment, autopoietism.

The encrypted text can be used to drive an oscillator to impart energy to a subject.

### Example 2: Religious Text and Music Delivered Via Vibroacoustic Noninvasive Stimulation (VATS) to Human Coronary Endothelial Cells

Endothelial dysfunction is a hallmark of various cardiovascular diseases. Endothelial Cells (EC) mechano-sensor-proteins Syndecan-4 (Syn4), VEGF and KLF2 can translate physical force from vascular microenvironment into biochemical signals. Syndecan-4 is a ubiquitous cell surface proteoglycan, mediates numerous cellular processes through signaling pathways which affect cellular mechano-transduction, proliferation and migration. Syndecan-4 functions as an independent receptor for heparin-binding growth factors, such as vascular endothelial growth factors (VEGFs). These signaling cascades affect signaling components, such as AKT and eNOS. Syndecan-4 is also able to form physical connections between the extracellular matrix (ECM) and cytoskeletal signaling proteins. KLF2 regulates the eNOS promoter and acts as eNOS transcription factor increasing its expression. Nitric oxide production in response to mechanical forces applied on the surface of endothelial cells is a fundamental mechanism of regulation of vascular tone, peripheral resistance, and tissue perfusion.

This implicates the concerted action of multiple upstream “mechano-sensing” molecules recruiting endothelial nitric oxide synthase (eNOS). Endothelial expression of syndecan-4 also increases VEGF-induced NO release. Subsequent short- and long-term increases in activity and expression of eNOS translate mechanical stimulus into enhanced NO production and bioactivity through eNOS regulatory phosphorylation, and protein-protein interactions. eNOS expressed in cardiac myocytes in part mediates the length-dependent increase in cardiac contraction force, short-term regulation of contractile tone, and cardiac and vascular remodeling. KLF2, Syn4 and VEGF also mediate eNOS phosphorylation which results in higher levels of p-eNOS/NO and inhibition of endothelial adhesion molecules during endothelial injury.

We set out to compare Psalm (Religious Text+Music) vs. Song (Regular Text+Music) VATS stimulation of mechano-sensors of human coronary endothelial cells—Syn4, VEGF and KLF2, which will induce eNOS expression and phosphorylation. For songs we played I Will Survive by Gloria Gaynor, Happy by Farrell Williams and Un-break My Heart by Tony Braxton. The religious text and music were Psalms 13, 17 and 20 as taken from these You tube links: https://youtu.be/NBOeCekvfO0; https://youtu.be/QlxeBkzljhA; https://youtu.be/yXcILO5lFao. These were played continuously during the study.

The above songs and Psalms were process through the Solfa Cipher program that turns text into singable melodies and the melodies used to drive the VATS. As a control a 100 hz sinusoidal wave was administered.

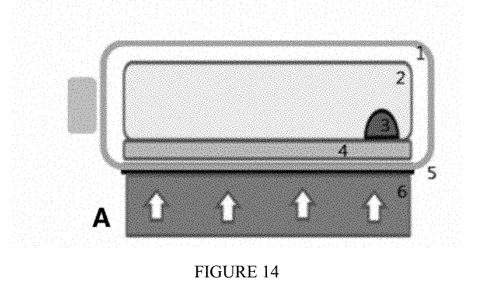

Referring to FIG. 14, a horizontal culture of Human Coronary Endothelial Cells (HCEC) was treated by Psalms or Song or a 100 hz sinusoidal wave control via VATS corresponding to shear stress forces in human coronary arteries at an energy level of 20 dyn/cm²; for 2 days, 2 times a day, 30 min each. HCEC were incubated at 37° C. and humidified 5% CO2/95% air. Levels of Syn4, VEGF, KLF2, eNOS and p-eNOS in the cell lysate were analyzed using western blot and fluorescent scanning.

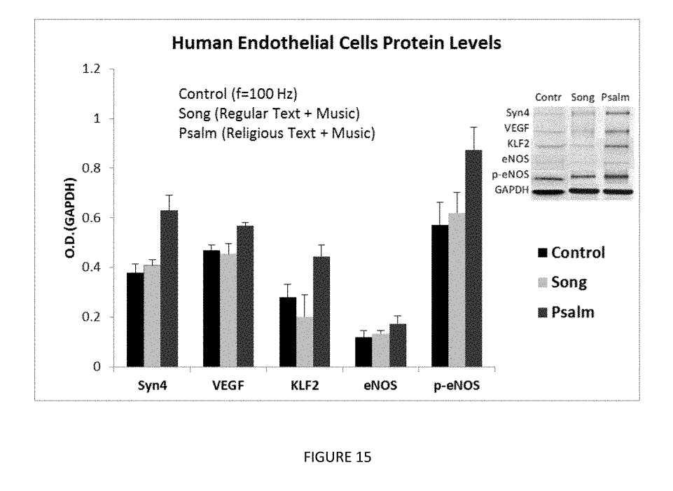

Referring to FIG. 15, Psalms significantly increased Syn4, VEGF, KLF2 levels by 35, 20, and 55% respectively compare to Song stimulation or the control. Levels of eNOS and p-eNOS were also elevated after 2 days after Psalm stimulation by 22 and 29%.

To our great and unexpected surprise, Psalm (Religious Text and Music) delivered via VATS stimulation of HCEC dramatically increases eNOS and p-eNOS possibly via special preprogramed “hard-wired” cellular mechano-sensors mechanism. These studies support the unexpected concept of noninvasive modulation of vascular biology using Religious Text and Music and development of a new noninvasive wearable therapy to improve endothelial dysfunction.

### Example 3: Religious Text and Music Delivered to Carotid Arteries Via Wearable Vibroacoustic Stimulation (VATS)

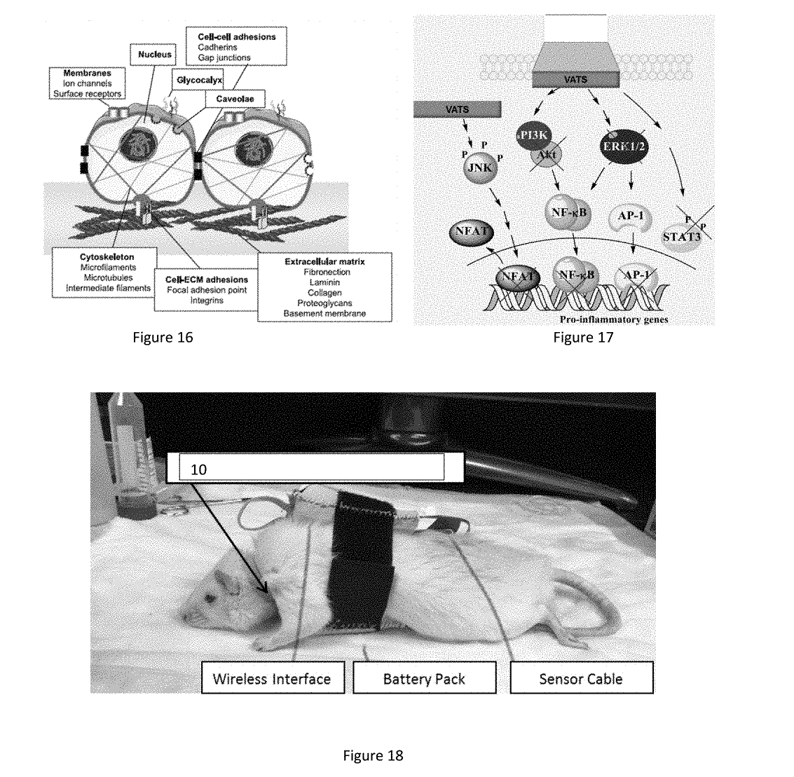

We set out to compare Psalm (Religious Text+Music) vs. Song (Regular Text+Music) VATS induction of Syndecan-4 mechano-sensor cardioprotective control of NFAT and JNK/ERK delivered to Carotid Arteries in Rats. See FIGS. 16 and 17.

Male Sprague Dawley rats (300 g) were randomized into three groups. Group 1 (n=5) received 1 hr Psalms daily via VATS (MI-Psalm) for 4 weeks. Group 2 (n=5) received 1 hour of non-religious music daily (MI-Song) for 4 weeks. A control surgery group (n=5) received a 100 hz sinusoidal wave. Left ventricular (“LV”) fibrosis and levels of NFAT, JNK, ERK, Syn4 proteins after myocardial infarction (“MI”) with or without Psalm/Song VATS were analyzed. Referring to FIG. 18, the oscillator 10 was placed on the carotid arteries of the rats and driven by a wireless signal.

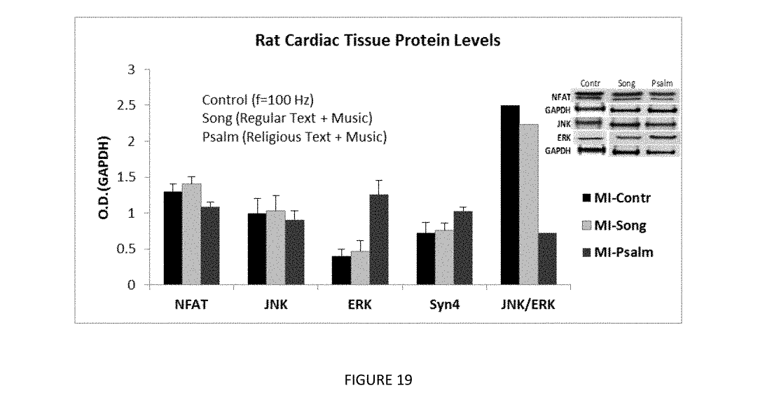

Referring to FIG. 19 religious text via VATS after MI reduced left ventricular fibrosis by 19% [MI-Song 36(4)% vs MI-Psalm 24(3)% (p<0.01) as a % of the LV]. JNK/ERK ratio and NFAT levels were lower in LV of MI-Psalm rats by 63% and 30% respectively when compared to MI-Song (p<0.01) that contributed to LV fibrosis reduction by 19% (p<0.01). Religious Text+Music significantly increased Syn4 and LV function (p<0.01, MI-Psalm vs MI-Song).

To our great and unexpected surprise in a rat model heart failure induced by chronic MI, a wearable Vibroacoustic Carotid Stimulator (VATS) transmitting Religious Text+Music (Psalm) provided better reduction of fibrosis compare to regular Song or control. Religious Text can deliver unexpected benefits possibly via special preprogramed “hard-wired” cellular modulation of Syndecan-4 expression. Our findings suggest that after MI Religious Text delivered via VATS noninvasively improves LV morphology and function via mechano-sensor modulation of NFAT, JNK and ERK, possibly via unique transcriptional mechanism. These findings have implications for Novel cardiac rehabilitation post myocardial infarction.

### Example 4: Placement of Oscillators at Points of Religious Significance

In addition to placing the oscillators 10 over arteries, the transducers can also be located on the body in places of religious significance or in religious symbols which are used to touch the body. Religious symbols can be selected from books, clothing, drinking vessels or any object which has religious significance to someone being treated.

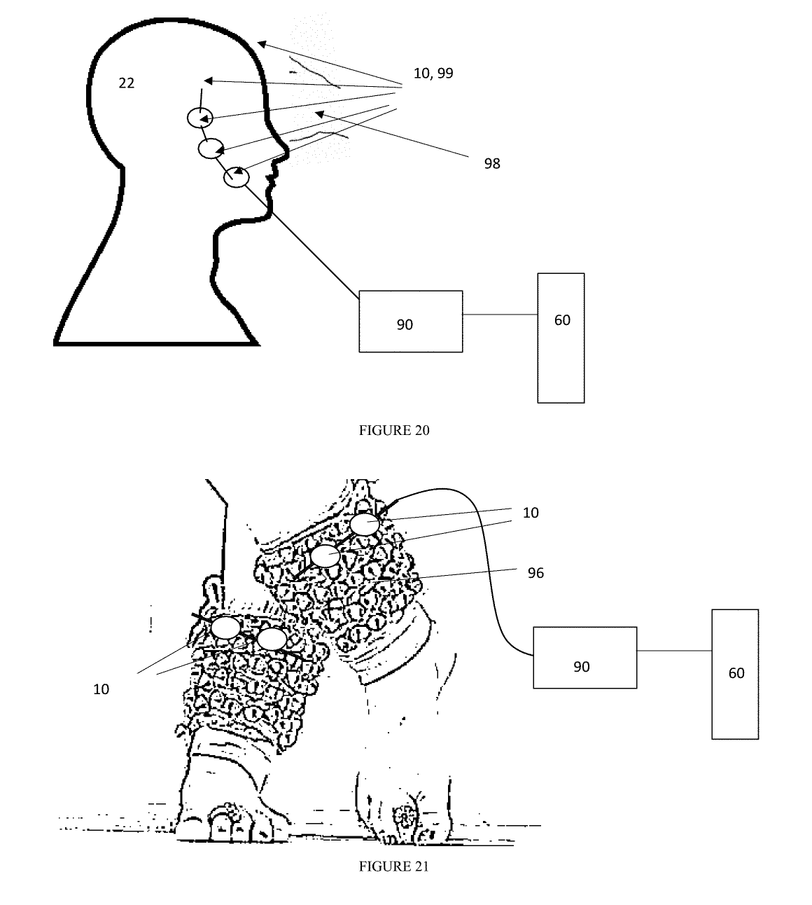

Referring to FIG. 20, one or more oscillators 10 can be placed on the head 22 of a user to mimic a Jewish or Christian laying of hands 98. The oscillators 10 can either be placed directly on the head 22 or on the hands 98 of a person who is touching the person receiving the therapy. When placed on the hands 98, the oscillators 10 can be placed on the finger tips, palms or back of the hands. The oscillators are driven by MUSART processor 90 which in this embodiment receives source material from cell phone 60. In one embodiment a visual display depicting at least one of Jesus, an apostle, a prophet and a rabbi, performing said laying of hands is administered to said subject during said audio haptic experience.

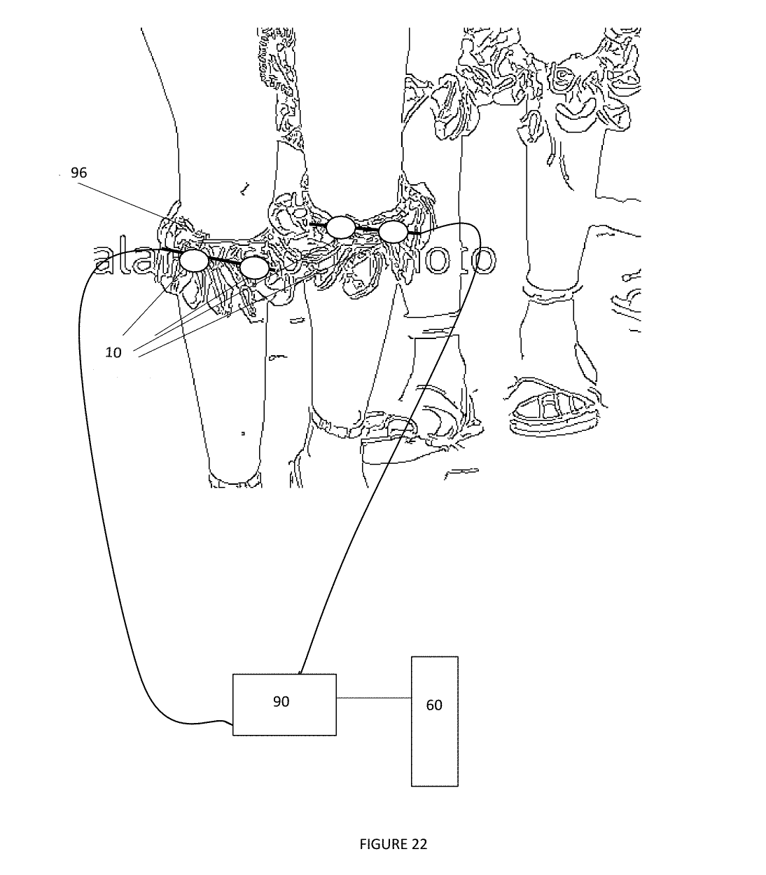

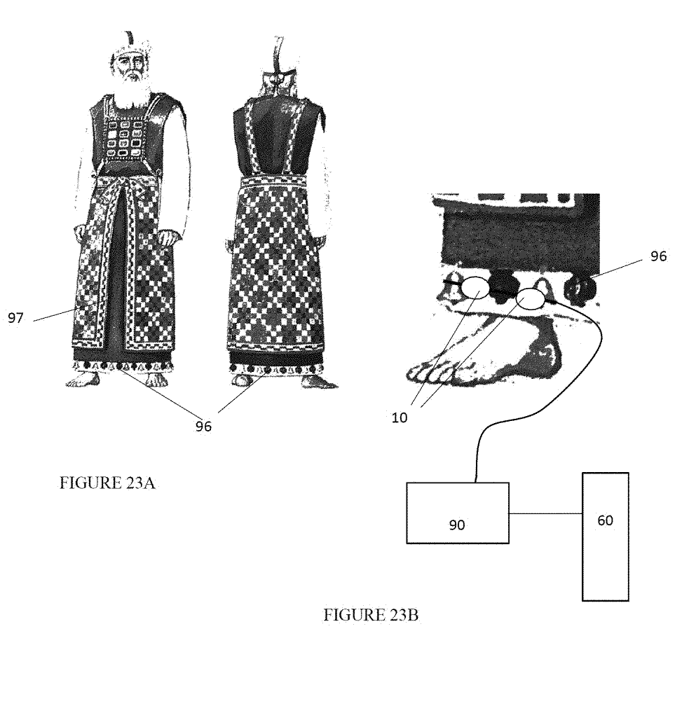

Referring to FIGS. 21, 22 and 23A and 23B, bells 96 are frequently worn in many faiths. FIG. 21 shows Hindu bells 96 worn on the ankles. FIG. 22 shows Muslim bells 96 worn about the calves. FIG. 23A shows bells 96 worn on the hem of a Jewish High Priest. FIG. 23B shows that Oscillators 10 can be worn in place of bells for therapeutic purposes. The oscillators are driven by MUSART processor 90 which in this figure receives source material from cell phone 60.

As will be immediately apparent to those skilled in the art in light of the foregoing disclosure, many alterations and modifications are possible in the practice of this invention without departing from the spirit or scope thereof. Accordingly, the scope of the invention is to be construed in accordance with the substance defined, and as described, by the following claims.

We claim:

1. A method to enhance healing or therapy in a living organism in need thereof, comprising the steps of:
a) providing an audio signal comprising religious text involving the subject matter of healing;
b) generating a sound from said audio signal wherein the sound comprises a recitation of said religious text, said sound supplied to the ears of a subject requiring healing or therapy; and
c) generating a tactile oscillation having a waveform synchronized in cadence and frequency with said sound, said tactile oscillation administered via a mechanical actuator placed in contact with said subject, said actuator and placement applied at a sufficient intensity that such subject feels the tactile oscillation;
whereby the subject hears the sound of recitation of religious text while feeling said tactile oscillation synchronized in cadence and frequency with said sound.

2. The method of claim 1 wherein the religious text is converted via music cryptography to musical notes.

3. The method of claim 1 where the actuator is placed upon a palpable artery of the subject or upon the backside, palms or fingers of a hand or a replica of a hand.

4. The method of claim 3, where said the religious text is selected from at least one of the of the following religions: Christianity, Judaism, Hinduism, Islam, Buddhism.

5. The method of claim 4 wherein the religious text is a Psalm.

6. The method of claim 5 wherein the Psalm specifically addresses healing.

7. The method of claim 1, wherein a bass frequency of said tactile oscillation waveform emitted by said mechanical oscillator is amplified relative to higher pitched frequencies.

8. The method of claim 7, wherein said tactile oscillation waveform is combined with a second oscillation waveform having a waveform frequency in the 1 Hz to 300 Hz range emitted by said mechanical oscillator during moments of inactivity or silence with respect to the emission of said audible sound comprising said recitation of religious text.

9. The method of claim 3, wherein an arterial locator is disposed along-side said mechanical oscillator to assist in placement of said mechanical oscillator upon a palpable artery.

10. The method of claim 4 wherein the organism is a mammal.

11. The method of claim 10 wherein the mammal is human.

12. The method of claim 1 wherein the patient has a disease having a fibrotic or inflammatory component wherein Syn4, VEGF, KLF2 are increased at least 20% over administration of non-religious songs.

13. The method of claim 1 wherein JNK/ERK ratio is lowered by about 63 percent and NFAT levels are lowered by about 30 percent in the left ventricle of a heart that has suffered a myocardial infarction compared to treatment by non-religious text.

14. The method of claim 12 wherein the disease is selected from: treatment of arthritis, treatment of inflammation, treatment of pain, treatment of erectile dysfunction, treatment of a broken bone, treatment of ischemic disease, treatment of refractory angina, treatment of thrombosis, promoting cerebral vascular angiogenesis, promoting coronary angiogenesis, enhancing mental acuity, and enhancing athletic performance

15. The method of claim 1 wherein the oscillator is placed on the subject at a location of religious significance.

16. The method of claim 1 wherein the oscillator is placed on an object which is used to make contact with a subject.

17. The method of claim 16 wherein the object is a religious symbol.

18. The method of claim 17 wherein the religious symbol is selected from books, clothing, or drinking vessels.

19. The method of claim 18 wherein the religious symbol is a holy book.

20. The method of claim 18 wherein the oscillator is located in a religious garment

21. The method of claim 20 wherein the oscillator is worn in place of religious bells or noisemakers.
